(19) United States
(12) Patent Application Publication
(10) Pub. No.: US 2022/0233130 A1
(43) Pub. Date: Jul. 28, 2022

(54) APPARATUS AND PROCESS FOR MEASURING BRAIN ACTIVITY

(71) Applicant: Cortical Dynamics Limited, Western Australia (AU)

(72) Inventor: David Tibor Julian LILEY, Western Australia (AU)

(21) Appl. No.: 17/614,701

(22) PCT Filed: May 28, 2020
(86) PCT No.: PCT/AU2020/050535

§ 371 Date: Nov. 29, 2021

(30) Foreign Application Priority Data
May 28, 2019 (AU) 2019901839

(51) Int. Cl. A61B 5/372; A61B 5/291; A61B 5/00

(52) U.S. Cl. CPC A61B 5/372; A61B 5/291; A61B 5/7225; A61B 2505/05; A61B 5/7267; A61B 5/7275; A61B 5/4821; A61B 5/7239

(57) ABSTRACT

A computer-implemented process for measuring brain activity of a subject, including: receiving electroencephalogram (EEG) data representing EEG measurements of electrical brain activity of the subject; and fitting parameters of a neural population model to the EEG data to determine corresponding values of said parameters, including one or more values of an inhibitory postsynaptic potential rate constant (γᵢ), where γᵢ is representative of states of brain responsiveness, an increase in γᵢ relative to a reference value indicating an enhanced state of brain responsiveness, and a decrease in γᵢ relative to the reference value indicating a decrease in the state of brain responsiveness.

### TECHNICAL FIELD

The present invention relates to an apparatus and process for measuring brain activity based on electroencephalography (EEG) signals.

### BACKGROUND

Electrical activity in the brain is a result of the flow of charge induced by neuronal activity, and varies in distinctive patterns (such as a “wave”) across adjacent brain regions in response to stimuli. The overall synchronous activity of neurons in a human brain can be recorded using Electroencephalography (EEG), which involves the use of electrodes to measure electric potentials on the outside of the scalp of an individual (and hence infer neuronal function).

EEG provides a simple and non-invasive way to study brain activity based on particular features that appear in the signal output. EEG signals often contain rhythmic features occurring in particular frequency bands, including those referred to in the art as the alpha, beta, theta, and delta bands. The alpha rhythm feature has long been considered to be an indicator of brain information processing and function. Specifically, the characteristic oscillations of the alpha rhythm at 8-13 Hz have played a central role in phenomenological descriptions of brain electromagnetic activity during cognition and in behaviour.

The presence of characteristic features in EEG signals motivates the use of EEG signal analysis for brain function monitoring applications, including seizure diagnosis and treatment (e.g., to distinguish epileptic seizures from other types of seizure, and to identify the region of the brain in which a seizure has originated), and determination of the depth of anaesthesia of an individual (e.g., during surgery).

Physiological interpretation of features in EEG signals has often involved use of collective models of neural populations, referred to in the art as “neural population models”, which represent the large numbers of interacting neurons as a macroscopic system. Neurophysical modelling, which is concerned with modelling the structure of the human brain, has utilised neural population models in an attempt to understand and characterise the relationship between features of EEG signals observed from an individual and activity within the individual's brain.

Specifically, a neural population model includes a set of parameters describing the excitatory and inhibitory neuron populations, and where the values of these parameters determine the predicted electrical response of the modelled neurons to particular stimuli and inputs. The predicted EEG signals produced by these neural population models can reproduce prominent features of the EEG, such as, for example, the alpha-rhythm.

Despite the convenience of conventional means of performing brain activity measurement, or monitoring, based on neurophysical modelling technologies, there remains room for improvement. It is desired to provide a brain activity assessment apparatus and process that alleviate one or more difficulties of the prior art, or to at least provide a useful alternative.

### SUMMARY

In accordance with some embodiments of the present invention, there is provided a computer-implemented process for measuring brain activity of a subject, the process including: receiving electroencephalogram (EEG) data representing EEG measurements of electrical brain activity of the subject; and fitting parameters of a neural population model to the EEG data to determine corresponding values of said parameters, including one or more values of an inhibitory postsynaptic potential rate constant (γᵢ); wherein γᵢ is representative of states of brain responsiveness, an increase in γᵢ relative to a reference value indicating an enhanced state of brain responsiveness, and a decrease in γᵢ relative to the reference value indicating a decrease in the state of brain responsiveness.

In some embodiments, the steps of the process for measuring brain activity of the subject are repeated in real-time to determine values of γᵢ for respective times to provide real-time monitoring of the brain responsiveness of the subject.

In some embodiments, the EEG signals are measured during sedation of an unstimulated subject, and the determined values of γᵢ for respective times quantify the depth of sedation of the subject, wherein a decrease in γᵢ relative to the reference value indicates a decrease in brain responsiveness or wakefulness of the unstimulated subject.

The unstimulated subject may be anesthetized, or undergoing a surgical procedure.

In some embodiments, the reference value is a predetermined measurement of γᵢ in a control subject or a plurality of control subjects.

In some embodiments, the reference value is a value of γᵢ determined by fitting the neural population model to EEG measurements of the subject in a state of wakefulness or awareness.

In some embodiments, the reference value is a predetermined value of γᵢ that is representative of brain activity in a control subject or plurality of control subjects at a level of sedation selected from a group consisting of: minimal sedation (Stage I), conscious sedation (Stage II), deep sedation (Stage III) and medullary depression (Stage IV).

In some embodiments, an anaesthetic has been administered to the subject, and the processes described above include:
(i) comparing a value of γᵢ to the reference value; and
(ii) determining whether the subject requires additional anaesthesia on the basis of said comparing; and
(iii) only if it is determined that the subject requires additional anaesthesia, then:

administering additional anaesthesia to the subject.

The subject may be undergoing a surgical procedure.

In some embodiments, the additional anaesthesia is delivered by an automated anaesthetic delivery system.

In some embodiments, the one or more values of γᵢ quantify alpha-rhythmic features of the brain activity of the subject.

In accordance with some embodiments of the present invention, there is provided a system for measuring brain activity in a subject, including:

at least one network interface configured to receive data from a communications network;

at least one computer processor; and

a memory coupled to the at least one computer processor and storing instructions that, when executed by the at least one computer processor, cause the at least one computer processor to execute any of the brain activity measurement processes described above.

In accordance with some embodiments of the present invention, there is provided an apparatus for measuring brain activity in a subject, including:
at least one computing device having a neural population model fitting component configured to:
(i) receive EEG data representing EEG measurements of electrical brain activity of the subject; and
(ii) fit parameters of a neural population model to the EEG data to determine one or more values of said parameters, including one or more values of an inhibitory postsynaptic potential rate constant (γᵢ);
   wherein γᵢ is representative of states of brain responsiveness, an increase in γᵢ relative to a reference value indicating an enhanced state of brain responsiveness, and a decrease in γᵢ relative to the reference value indicating a decrease in the state of brain responsiveness.

In some embodiments, the neural population model fitting component is configured to execute any of the brain activity measurement processes described above.

In some embodiments, the apparatus includes an EEG monitoring component configured to:
generate the EEG data by processing EEG signals obtained from one or more electrodes; and
transmit the EEG data to the neural population model fitting component.

In accordance with some embodiments of the present invention, there is provided one or more computer-readable storage media having stored thereon instructions that, when executed by at least one processor of a computing system or device, cause the at least one processor to execute any of the brain activity measurement processes described above.

Also described herein is a process of identifying physiological parameters for assessing brain function, including: receiving EEG data representing the electrical brain activity of one or more individuals; accessing neural population model data representing a neural population model having one or more model parameters; generating, according to a model fitting process which fits the one or more model parameters to the EEG data, fitted model parameter data representing estimated parameter values for the one or more model parameters; processing the fitted model parameter data to generate parameter identifiability data representing, at least, the individual identifiability of each model parameter; and processing the parameter identifiability data to generate determinative parameter data representing an indication of one or more determinative parameters that characterise one or more features of the EEG data, such that the values of the determinative parameters provide an indication of corresponding brain function of an assessed individual.

In some implementations, generating the parameter identifiability data includes, for each parameter: i) estimating a marginal posterior distribution of the parameter; and ii) determining a difference in variability of the parameter between its prior and posterior distributions.

In some implementations, the Kullback-Leibler divergence is used to measure the difference in variability of the parameter.

In some implementations, the parameter identifiability data additionally represents the collective identifiability of the model parameters.

In some implementations, the collective identifiability of the model parameters is determined from an Eigen-analysis of the Fisher Information Matrix derived from the parameters.

In some implementations, the identifiability of the model parameters is processed to produce an indication of model sloppiness representing the sensitivity of predictions of the neural population model to variations across the one or more model parameters.

In some implementations, the determinative parameter data is processed to generate optimised neural population model data representing a new neural population model with a reduction in the degrees of freedom.

In some implementations, the neural population model is described by a coupled set of 1st and 2nd order ordinary differential equations.

In some implementations, the neural population model defines the interactions between inhibitory and excitatory neuronal populations based on a set of physiological parameters.

In some implementations, the physiological parameters include one or more measures of: resting membrane potential; membrane potential; mean firing rate; firing thresholds; passive membrane decay time constant; excitatory postsynaptic potential rate constant; inhibitory postsynaptic potential rate constant; postsynaptic potential amplitude; rate of the excitatory or inhibitory input to either population; and the total number of connections an excitatory or inhibitory neuron receives from either nearby excitatory or inhibitory neurons.

In some implementations, the determinative parameter set includes a measure of the dynamics of inhibitory synaptic activity of the one or more individuals, as represented by at least one postsynaptic inhibitory rate constant parameter, for characterising an alpha-rhythmic feature of the EEG data.

In some implementations, the model fitting process involves the steps of: accessing prior distribution data describing an assumed distribution for each parameter; determining estimation criterion functions to assess candidate parameter values based, at least in part, on the EEG data; and generating, according to a fitting algorithm and the estimation criterion functions, sample value data representing, at least, the estimated parameter values.

In some implementations, the fitting algorithm is a particle swarm optimisation (PSO) algorithm, and where the estimated parameter values are derived based, at least in part, on minimisation of a least squares cost function.

In some implementations, the fitting algorithm is a Markov Chain Monte Carlo (MCMC) algorithm, and where the estimated parameter values are derived by maximising the likelihood function of the parameters.

In some implementations, the EEG data is processed prior to performing the model fitting process to generate at least one EEG spectrum.

In some implementations, the at least one EEG spectrum is produced by averaging spectra derived from overlapping time series EEG signals of the one or more individuals.

In some implementations, the estimation criterion functions include: i) a predicted model spectrum function of the neural population model; and ii) a corresponding likelihood function for the predicted model spectrum function, and where the estimated parameter values are derived based on the at least one EEG spectrum and the estimation criterion functions.

Also described herein is a system configured to perform neurophysical assessment, including: (i) a data handler to: receive EEG data representing the electrical brain activity of one or more individuals; access neural population model data representing a neural population model having one or more model parameters; (ii) a sampling and estimation unit to generate, based on a model fitting process which fits the one or more model parameters to the EEG data, fitted model parameter data representing estimated parameter values for the one or more model parameters; and (iii) a parameter analyser to: process the fitted model parameter data to generate parameter identifiability data representing, at least, the individual identifiability of each model parameter; and process the parameter identifiability data to generate determinative parameter data representing an indication of one or more model parameters that characterise one or more features of the EEG data.

Also described herein is a process for assessing brain function during a surgical procedure performed on a patient, wherein the assessment of brain function is based on the generation of values for one or more parameters determined from a neurophysical model, wherein the parameters are determined according to the process of physiological parameters identification as described herein.

Also described herein is a process for assessing brain function during the administration of anaesthesia to an patient, wherein the assessment of brain function is based on the generation of values for, at least, an inhibitory postsynaptic potential rate constant parameter of a neurophysical model, the inhibitory postsynaptic potential rate constant parameter characterising an alpha-rhythmic feature of electroencephalogram (EEG) data for human individuals.

### BRIEF DESCRIPTION OF THE DRAWINGS

Some embodiments of the present invention are hereinafter described, by way of example only, with reference to the accompanying drawings, wherein:

FIG. 1 is a block diagram of a neurophysical assessment apparatus in accordance with some embodiments of the present invention;

FIG. 2 is a block diagram of a computing device of the neurophysical assessment apparatus;

FIG. 3a is a flow diagram of a process for performing parameter identification for neurophysical assessment in accordance with some embodiments of the present invention;

FIG. 3b is a flow diagram of a process for determining brain function for neurophysical assessment in accordance with some embodiments of the present invention;

FIG. 4 is a flow diagram of a model fitting process of the neurophysical assessment process of FIG. 3;

FIG. 5 is a flow diagram of a process for generating parameter identification data of the neurophysical assessment process;

FIG. 6 is a schematic block diagram of a feedback system for producing a predicted model spectrum during model fitting for the neurophysical assessment process;

FIG. 7 is a set of graphs of power spectra density as a function of frequency for six subjects, illustrating best fits of model parameters using least-squares based estimation for the neurophysical assessment process;

FIG. 8 is a set of graphs of power spectra density as a function of frequency for six subjects, illustrating best fits of model parameters using maximum likelihood based estimation for the neurophysical assessment process;

FIG. 9 is a set of graphs showing posterior distributions for each parameter for seven subjects as generated based on particle swarm optimisation (PSO) sampling for the neurophysical assessment process;

FIG. 10 is a set of graphs showing posterior distributions for each parameter for seven subjects as generated based on Markov Chain Monte Carlo (MCMC) sampling for the neurophysical assessment process;

FIG. 11 is a plot showing the values and distributions of Kullback-Leibler divergences for each parameter determined from fitting based on particle swarm optimisation (PSO) sampling for the neurophysical assessment process;

FIG. 12 is a plot showing the values and distributions of Kullback-Leibler divergences for each parameter determined from fitting based on Markov Chain Monte Carlo (MCMC) sampling for the neurophysical assessment process;

FIG. 13 is a graph showing the Fisher Information Matrix (FIM) eigenspectra generated based on best fits of model parameters using least-squares based estimation for the neurophysical assessment process;

FIG. 14 is a graph showing the FIM eigenspectra generated based on best fits of model parameters using maximum likelihood based estimation for the neurophysical assessment process;

FIG. 15 is a graph of FIM eigenvector components corresponding to first, second and third eigenvalues based on best fits of model parameters using least-squares based estimation for the neurophysical assessment process; and

FIG. 16 is a graph of FIM eigenvector components corresponding to first, second and third eigenvalues based on best fits of model parameters maximum likelihood based estimation for the neurophysical assessment process.

### DETAILED DESCRIPTION

### Overview

There is an important clinical need to be able to measure brain activity in a subject, particularly in real-time during a medical intervention such as surgery, and to monitor the level of anaesthesia during surgery. Features of EEG signals from human individuals are related to their brain activities. For example, the alpha rhythm is a defining feature of the human resting EEG signal, and has played a central role in phenomenological descriptions of brain electromagnetic activity during cognition. A macroscopic approach to assessing brain function (referred to herein as “neurophysical assessment”) via a characterisation of EEG features, such as the alpha-rhythm, is based on a neural population model. Conventionally, the neural population model parameters are determined by iterative tuning methods, where the best parameters are chosen from the tuning set as those for which the model produces (or “predicts”) an EEG signal that exhibits a particular feature of an observed EEG signal (e.g., particular alpha oscillations). The assumption is that a matching of features between the predicted and observed EEG signals indicates model parameter values that accurately, and thus meaningfully, represent neuronal activity.

However, neural population models are high-order, multi-parameter, dynamical systems, and as such different model parameter combinations can generate not only similar predictions, but in many cases identical predictions. That is, there is a many-to-one mapping between combinations of the neural population model parameter values and resulting features in the predicted EEG signals. This many-to-one mapping between parameter inputs and model observables is referred to in the art as structural unidentifiability, if the predictions are exactly identical, or “practical unidentifiability”, if the predictions are nearly identical. As a consequence, a drawback of conventional neural population model parameter tuning is that they are prone to producing structurally unidentifiable models that, when fitted to data, result in large correlated parameter uncertainties for variations performed to the parameter values.

Parameters that sensitively affect model predictions are termed “stiff” while those that can be changed with little effect on predictions are termed “sloppy”. A further drawback of tuning based approaches is that the resulting neural population model may include a large number of “sloppy” parameters. As a result, the sensitivity of the predictions of the neural population model can be undesirably affected such that the values of the various parameters (or combinations of parameters) can be estimated with varying degrees of certainty and thus accuracy.

Compared to conventional neural model parameter tuning, a more desirable approach would be to obtain the model parameters directly from the observed EEG data for a given individual. Being able to estimate these parameters by direct fits to EEG data holds the promise of providing a real-time non-invasive method of inferring neuronal properties in different individuals, such as for the purpose of assessing their brain function. Despite this promise, it is vastly more difficult to solve the problem of estimating a full set of parameters and their uncertainties directly from fits to EEG data, as compared to parameter tuning. However, it has not previously being recognized that determining which parameters (or parameter combinations) are “stiff” and which are “sloppy” can be beneficial in order to usefully fit these nonlinear multi-parameter neural models to real EEG data.

The described embodiments of the present invention include an apparatus and process for measuring brain activity in a subject, in which a neural population model is fitted to EEG data collected from a subject to determine corresponding values for an inhibitory postsynaptic potential rate constant (γᵢ), these values constituting quantitative measures of brain activity of the subject. In the described embodiments, the inhibitory postsynaptic potential rate constant (γᵢ) was identified by a model fitting process that involves finding the particular parameters of the model which are identifiable for the neuronal population model using a fitting process based on group data (i.e. data obtained from a training or validation group). An assessment of brain function of a subject is then able to be performed based on EEG data obtained from the subject in real-time.

Specifically, the presented apparatus includes a neurophysical assessment component that receives EEG data representing the electrical brain activity of one or more individuals. Neural population model data is accessed, where the data represents a neural population model having particular parameters. The received EEG data is then processed in order to determine neuronal population model parameters (e.g., by estimating parameter values for the one or more model parameters in view of the data). The resulting model is then processed to generate data representing the individual identifiability of each model parameter (e.g., based on variability between prior distributions and corresponding posterior distributions). The parameter identifiability data is then used to identify the “determinative” model parameters, these being the parameters that are both identifiable and “stiff” in the sense of characterising one or more features of the EEG data (e.g., the alpha-rhythm).

Given an indication of the determinative parameters, the neurophysical assessment component determines the brain function (or “activity”) of a subject (e.g. a patient undergoing a surgical procedure) based on EEG data measured from the subject. Estimates of the values of the determinative parameters are produced by fitting the neural population model to the measured EEG data (unlike conventional approaches that rely on a direct analysis of the EEG signal with respect to the feature of interest).

In some embodiments, the neurophysical assessment component receives measured EEG data, and fits the neural population model to the data to produce determinative parameter value estimates, in real-time. Following model fitting, the values of the determinative parameters can be processed to provide an indication of corresponding brain activity of the subject. Although the neurophysical assessment component described herein calculates the determinative parameters via the model fitting process, in other embodiments the neurophysical assessment component may be configured to perform the brain activity measurement based on prior knowledge of the determinative parameters (i.e., without conducting the parameter identification sub-process).

An application of the presented apparatus is described in which the postsynaptic inhibitory rate constant parameter γᵢ is identified as determinative for the alpha-rhythmic feature of EEG signals, allowing estimates of γᵢ measured from a subject to be used as a functional indication of the subject's brain activity. In this application, a neurophysical model is fitted to EEG data exhibiting alpha-oscillations to resting state EEG recordings from 82 human subjects. The determinative parameters identified using this apparatus form a set that is of significantly reduced dimensionality compared to the original model parameter space (as described below). For example, by identifying the determinative parameters that characterise the particular EEG features, the presented apparatus and process allows for a real-time and non-invasive characterisation of the physiological properties that underpin brain activity in human individuals.

In the described embodiments, the identifiability of each parameter is determined by estimating a marginal posterior distribution of the parameter. The Kullback-Leibler divergence (KLD) is used to separate the information gained from the data from that which was already present in the prior (as described below). A measure of the individual identifiability of the parameter is produced by determining a difference in variability of the parameter between its prior and posterior distributions. For example, the presence of significantly less variability in the posterior distribution of an EEG feature (e.g., the alpha-rhythm) compared to its prior indicates that the parameter is identifiable with respect to that feature (e.g., rate constant associated with inhibitory synaptic activity, as shown herein below).

In the described embodiments, differences between marginal posterior and prior distributions are used to infer information about parameters individually. In some embodiments, the apparatus and process are configured to generate parameter identifiability data that additionally represents the collective identifiability of the model parameters. In such embodiments, the collective identifiability of the model parameters is determined from an Eigen-analysis of the Fisher Information Matrix derived from the parameters. That is, the apparatus uses an indication of the second order behaviour of the model to infer interactions among the parameters, where these interactions may not be determined from a consideration of only the marginal posterior of each parameter individually.

Eigen-analysis of the Fisher Information Matrix has the advantage of allowing the interrelations between parameters to be assessed without requiring estimation of the joint posterior distribution (which is likely to be extremely computationally intensive, especially for high dimensional parameter spaces). Using this approach, the apparatus can generate quantitative measures of identifiability for combinations of a plurality of model parameters. In some embodiments, the apparatus also generates measures of neural population model sloppiness, which represents the sensitivity of predictions of the neural population model in respective ones of the model parameters.

The determination of measures of parameter identifiability and sloppiness can be used to quantify the degree to which a model is over-parameterized. Specifically, the existence of correlations between parameter estimates suggests that model complexity can be reduced by grouping together, eliminating, or averaging subsets of model parameters. In this manner, the apparatus can be configured to process the determinative parameter data to refine the neural population model in order to produce a refined model with reduced degrees of freedom, but without compromising its predictive ability.

In the described embodiments, neurophysical assessment is performed with EEG signal recordings obtained from one or more different individuals. The EEG signal recordings can be selected to emphasise the spatial location of oscillatory brain activity that are relevant to the brain activity functions of the individuals. For example, the apparatus may be configured to receive signals taken from particular electrodes (e.g., those in the Oz, Cz or Pz positions) such as to emphasise a particular feature in the resulting recordings. The time series signal of the received EEG data, when segmented appropriately, is processed to generate a single EEG frequency spectrum for each of the one or more individuals at one or more spatial scalp locations. However, while the use of a single spectrum for each individual reduces the computational requirements of performing the fitting model operations, such a restriction is not necessary and in no way limits the applicability of our process to multiple spatial locations and spectra.

In the described embodiments, the EEG spectral signal representations are fitted to a neural population model characterised by a coupled set of 1st and 2nd order ordinary differential equations. This allows the neural population model to define the interactions between inhibitory and excitatory neuronal populations (i.e. neurons which have inhibitory and excitatory effects on the postsynaptic target respectively) based on a set of physiological parameters. These parameters can include, but are not limited to, for each excitatory and inhibitory population: resting membrane potential; synaptic reversal potentials; mean firing rate; firing thresholds; passive membrane decay time constant; postsynaptic potential amplitude; rate of the excitatory or inhibitory input to either population. Additional parameters may include, the excitatory postsynaptic potential rate constant, the inhibitory postsynaptic potential rate constant, and the total number of connections an excitatory or inhibitory neuron receives from either nearby excitatory or inhibitory neurons.

In the described embodiments, the fitted model data is generated via a model fitting process that is executed by the neurophysical assessment component in respect of the EEG data. As described above, an issue with conventional approaches to fitting a neural population model is that a wide range of parameter combinations may be consistent with the observed spectrum. A sampling based approach is implemented by the apparatus and process described herein, involving the generation of many instances of the parameter values based on a prior distribution of the respective parameter. The sampled values are determined for each parameter by a fitting process according to the prior distribution. Estimation criterion functions are determined to assess candidate parameter values during the fitting process based, at least in part, on the EEG data. As a result, the sample value data generated by the fitting algorithm converges towards parameter value distribution estimates that best fit to the observed EEG data.

The apparatus can be configured to utilise particle swam optimisation (PSO) or Markov Chain Monte Carlo (MCMC) fitting algorithms to produce the marginal posterior parameter distributions from the sampled parameter values (as described herein below). In other embodiments, alternative fitting algorithms may be implemented to generate marginal posterior parameter distributions (for example, by producing a sample set of values for each parameter, filtering or selecting from the sample set, and then estimating the posterior from the reduced set of values).

The apparatus and processes described herein provide a platform for neurophysical assessment that determines parameter values for a given neural population model from dynamically observed EEG data in real-time. The platform can be utilised to provide a clinician with an indication of particular parameters, or combinations of parameters, that are determinative of one or more characteristic features of the EEG observations. Consequently, by utilising EEG data including a particular feature of interest (or selecting this data as a subset of the received EEG data during processing), insight can be obtained into which particular parameters are best able to account for variability in the feature of interest. Once these determine parameters are known, a clinician is able to utilise the apparatus to perform brain function monitoring for a subject (e.g., in the context of a surgical procedure), since estimates of these determinative parameters, as obtained in real-time from EEG recordings measured from the subject, can be used to make inferences regarding brain activity.

For example, the majority of agents used to induce a state of surgical anaesthesia are thought to function by altering the time course of postsynaptic inhibition. Determining the postsynaptic inhibitory rate constant γᵢ by fitting a neural model to EEG data (as described herein) can provide a real-time process for quantifying the functional effects of anaesthesia, and thus improve upon standard methods of neurophysical assessment which are not built on an underlying theory of brain dynamics. The apparatus and processes described herein are also advantageous in providing a way to reduce the dimensionality of a neural population model, compared to the original model parameter space, based on determinations of the most identifiable parameters, resulting in an increased practicality of use for the model (i.e. since specified EEG features can be predicted without loss of accuracy but with a reduction in the degrees of freedom).

### Neurophysical Assessment Apparatus

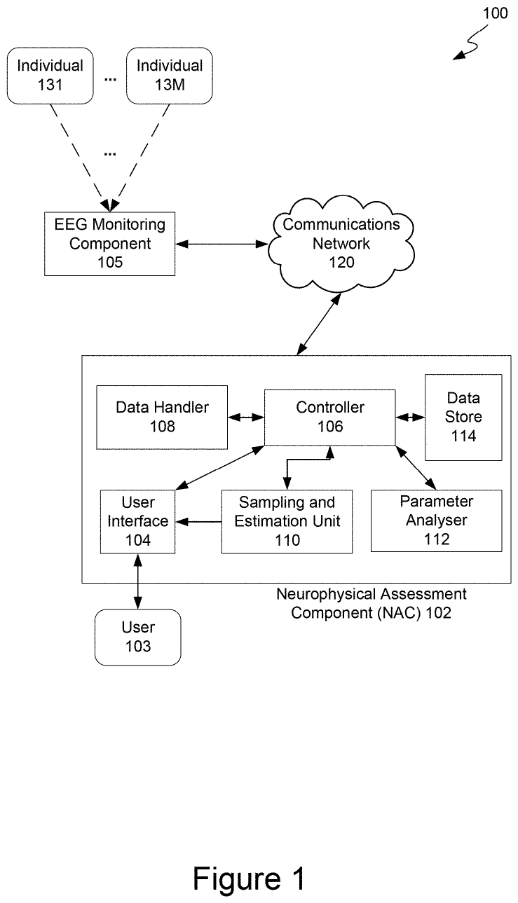

FIG. 1 is a block diagram of a neurophysical assessment apparatus 100 according to an embodiment of the present invention. The apparatus includes an EEG monitoring component 105 and a neurophysical assessment component 102. The EEG monitoring component 105 is configured to generate EEG signal data representing EEG signals of electrical brain activity of the one or more individuals 131-13M.

In some embodiments, the EEG monitoring component 105 includes: one or more electrodes, for measuring the electrical brain activity of the one or more individuals; and one or more associated EEG signal processing components (e.g., differential amplifiers, analog-to-digital converters, and anti-aliasing filters) for generating EEG recording data representing a time-domain signal of the recordings. In the described embodiments, the EEG monitoring component 105 outputs a set of EEG recordings for each individual. In some embodiments, the apparatus 100 does not include an EEG monitoring component 105 and EEG signal data is received from an external device (such as, for example, a third party EEG system).

In the described embodiments of the apparatus 100, the neurophysical assessment component (NAC) 102 is a computing device configured to execute a software application with modules including a user interface (UI) 104, a control unit 106, a data handler 108, a sampling and estimation unit 110, a parameter analyser 112, and a data store 114. In other embodiments, the aforementioned modules may be implemented by one or more dedicated hardware components of the device 102.

The NAC 102 is operated by a user 103, such as for example a clinician or other professional seeking to obtain diagnostic information in relation to neurological properties of individuals. User 103 interacts with the NAC 102 via UI 104. The UI 104 is configured to generate graphical user interface (GUI) data representing GUI elements that receive user input and display corresponding output (e.g., via one or more peripherals and a display of the computing device).

The controller 106 provides logical control and configuration functionality for the NAC 102. The controller 106 receives input and configuration data from UI 104 in respect of corresponding input and configuration instructions provided to the component 102 by the user 103. The controller 106 communicates with the data handler 108, sampling and estimation unit 110, and parameter analyser 112 to control the neurophysical assessment processes described herein (such as process 300) according to the options specified by the user 103.

Controller 106 is also configured to access EEG data, including a time domain representation of EEG recordings obtained from individuals 131-13M by the EEG monitoring component 105. In the described embodiments, the EEG data is received directly from the EEG monitoring component 105 via data transmission between the component 105 and the modelling component 102. In the described embodiments, the NAC 102 receives EEG data from the EEG monitoring component 105 via transmission over a communications network 120 using a secure communications protocol. In other embodiments, the monitoring component 105 is configured to transfer data to the component 102 via a direct connection (e.g. Ethernet over LAN).

The controller 106 can also be configured to retrieve previously stored EEG data for the one or more individuals 131-13M from the data store 114. Controller 106 transfers the received EEG data to and from the data store 114, and/or to the data handler 108, user interface 104, and sampling and estimation unit 110 to allow the neurophysical assessment processes described herein to be performed.

The data handler 108 performs signal analysis and processing operations on the EEG signal and/or the neural model data. In the described embodiments, the data handler 108 receives EEG data in the form of a time domain representation of EEG recordings (such as, for example, a discrete time series signal) for M individuals 131-13M. The data handler 108 performs operations which may include, but is not necessary limited to: spectral estimation; time and/or frequency averaging; and pre- or post-filtering.

The sampling and estimation unit 110 is configured to perform fitting between the processed EEG data and a neural population model with respect to particular parameters of the model. The neural population model, and the corresponding fitting algorithm, used by the unit 110 are determined based on control data provided by the control module 106.

Parameter analyser 112 is configured to produce measures of parameter identifiability and/or model sloppiness from fitted neural population model data. Analyser 112 is configured to generate data indicating particular determinative parameters, and/or parameter combinations, based on the produced measures and particular determinative parameter criteria. The determinative parameter criteria may vary in accordance with configuration options specified by the user 103 (as described below).

The data store 114 is configured to store data utilised by the component 102, including configuration data, neural model data, and EEG data. Configuration data specifies options and configuration variables of the component 102 for performing the neural modelling and evaluation processes described herein. In some embodiments, neural model data specifies, for each one of particular neural population models: the type of the model; the structural elements of the model (e.g., equations describing the model); and the parameters of the model. The neural model data also includes instances of fitted model parameter data, and parameter identifiability data, generated for the model. In some embodiments, the EEG data includes: time-domain representations of EEG recordings for, at least, individuals 131-13M, as received by the NAC 102; and processed EEG data resulting from the application of analysis or processing operations to the time domain data (e.g., averaged and/or spectral representations).

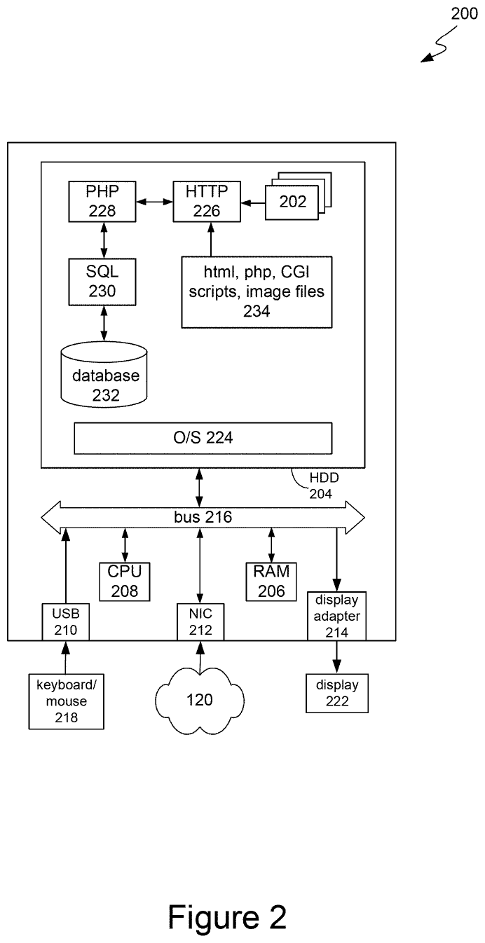

In the described embodiments of the apparatus 100, the NAC 102 is implemented as one or more computer systems 200, such as, for example, Intel Architecture computer systems, as shown in FIG. 2, and the processes executed by the system 200 are implemented as programming instructions of one or more software modules 202 stored on non-volatile (e.g., hard disk or solid-state drive) storage 204 associated with the computer system. However, it will be apparent that at least one or more parts of these processes could alternatively be implemented as configuration data of field programmable gate arrays (FPGAs), and/or as one or more dedicated hardware components, such as application-specific integrated circuits (ASICs), for example.

The system 200 includes random access memory (RAM) 206, at least one processor 208, and external interfaces 210, 212, 214, all interconnected by a bus 216. The external interfaces include universal serial bus (USB) interfaces 210, at least one of which is connected to a keyboard 218 and a pointing device such as a mouse 219, a network interface connector (NIC) 212 which connects the system 200 to the communications network 120, which may be a wide area network (such as the Internet), and a display adapter 214, which is connected to a display device such as an LCD or LED panel display 222.

The system 200 also includes a number of standard software modules 226 to 230, including an operating system 224 such as Linux or Microsoft Windows, web server software 226 such as Apache, available at http://www.apache.org, scripting language support 228 such as PHP, available at http://www.php.net, or Microsoft ASP, and structured query language (SQL) support 230 such as MySQL, available from http://www.mysql.com, which allows data to be stored in and retrieved from an SQL database 232.

In the described embodiments, the component 102 operates as a standalone computing device in which the database 232 is a local database managed by the data store component 114. The database 232 is implemented using SQL and is accessed by a database management system (DBMS) of the component 102. In other embodiments, the database 232 may be implemented on a separate computing device, or across multiple computing devices according to one or more techniques for the distributed processing and storage of data.

### Neurophysical Assessment Process

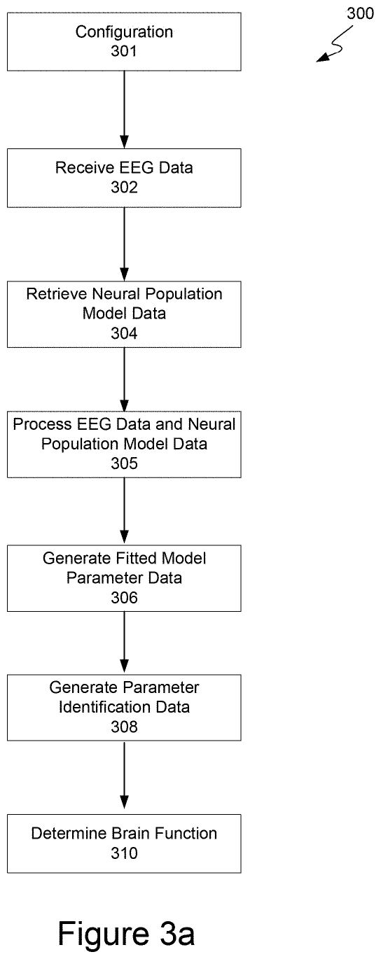

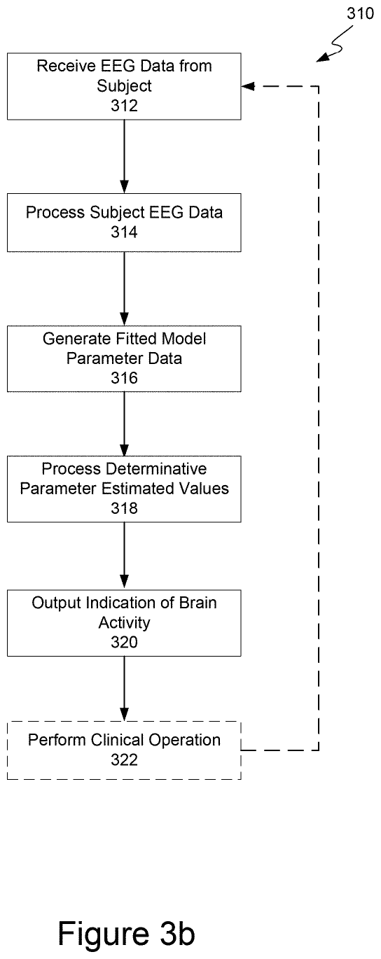

FIG. 3 illustrates a neurophysical assessment process 300 performed by the apparatus 100 to identify physiological parameters for assessing brain function. At step 301, preliminary configuration operations are performed for the monitoring 105 and modelling 102 components to initialise and/or configure the apparatus 100. Configuration of the monitoring component 105 may include the positioning of electrodes to gather electrical activity signals from individuals 131-13M and the setup of the processing devices to generate the EEG data from the electrode readings (i.e. during a clinical EEG recording procedure).

The configuration of the NAC 102 is performed by the user 103. Controller 106 receives user input and configuration data from UI 104 based on input obtained from the user 103. NAC configuration may include the user 103 specifying information in relation to particular system functions, including: the selection of EEG data; analysis and processing of the selected EEG data; neural population model and parameter prior distribution and/or fitting process selection; identifiability criteria for producing determinative parameter sets; and any refinement of the initial neural model based on the determinative parameters (e.g., reducing the number of parameters in the model).

In some embodiments, the selection of the EEG data to be used for modelling is based on the existence of one or more particular features of interest in the EEG signals represented by the data. In some embodiments, the UI 104 provides functionality allowing the user 103 to assess the suitability of particular received and/or stored EEG data for modelling operations (e.g., in the form of visualisation or display functions which illustrate the presence of EEG features, such as rhythmic components, in the recorded signals). In other embodiments, the apparatus 100 may be configured to perform neurophysical assessment automatically, based on real-time EEG data received from the monitoring component 105 (or other source), or retrieved from data store 114.

Note that although configuration is described as a preliminary step for the implementations discussed herein, in other implementations various functionality of the apparatus 100 can be configured at other times during the neurophysical assessment process 300. For example, selection of the EEG data to be used for modelling may be performed following at least some of the processing of the time-domain EEG data (i.e., to assess spectra characteristics, and subsequently select EEG data according to the presence, or otherwise, of particular spectra features).

At step 302, the EEG data is received by the NAC 102. In the described embodiments, the EEG data includes, for each individual 131-13M, separate time series recordings for each electrode of the monitoring component 105. At step 304, neural population model data is retrieved, from data store 114, in accordance with a selected neural population model that is to be fitted to the EEG data. The selected neural population model can be selected by the user 103 (e.g., during the configuration step 301), or determined automatically by the controller 106 (e.g., according to a default model setting).

Embodiments are described below for a neural population model corresponding to the default model of the NAC 102. The default model is the neural population model of Liley et al (Liley D T J, Cadusch P J, Dafilis M P. A spatially continuous mean field theory of electrocortical activity. Network 2002; 13:67). This well known model (see Coombes S. Large-scale neural dynamics: Simple and complex. NeuroImage 2010; 52:731) is described by a coupled set of first and second order ordinary differential equations given by Equations (1)-(6), where the meaning of the variables are given in Table 1 below. This model is implemented as the default model since semi-analytical and numerical solutions of the above equations have revealed a rich repertoire of physiologically plausible activity, including noise driven, limit cycle and chaotic oscillations in the frequency range of the mammalian alpha rhythm.

$${τ}_{e} \frac{{dh}_{e} ( t )}{dt} = {h}_{e}^{rest} - {h}_{e} ( t ) + \frac{{h}_{e}^{eq} - {h}_{e}}{\vert {h}_{e}^{eq} - {h}_{e}^{rest} \vert} {I}_{ee} ( t ) + \frac{{h}_{i}^{eq} - {h}_{e}}{\vert {h}_{i}^{eq} - {h}_{e}^{rest} \vert} {I}_{ie} ( t ) , \qquad ( 1 )$$

$${τ}_{i} \frac{{dh}_{i} ( t )}{dt} = {h}_{i}^{rest} - {h}_{i} ( t ) + \frac{{h}_{e}^{eq} - {h}_{i}}{\vert {h}_{e}^{eq} - {h}_{i}^{rest} \vert} {I}_{ei} ( t ) + \frac{{h}_{i}^{eq} - {h}_{i}}{\vert {h}_{i}^{eq} - {h}_{i}^{rest} \vert} {I}_{ii} ( t ) , \qquad ( 2 )$$

$$\frac{{d}^{2} {I}_{ee} ( t )}{{dt}^{2}} + 2 {γ}_{ee} \frac{{dI}_{ee} ( t )}{dt} + {γ}_{ee}^{2} {I}_{ee} ( t ) = {Γ}_{e} {γ}_{ee} e ( {N}_{ee}^{β} {S}_{e} ( {h}_{e} ) + {p}_{ee} ( t ) ) , \qquad ( 3 )$$

$$\frac{{d}^{2} {I}_{ie} ( t )}{{dt}^{2}} + 2 {γ}_{ie} \frac{{dI}_{ie} ( t )}{dt} + {γ}_{ie}^{2} {I}_{ie} ( t ) = {Γ}_{i} {γ}_{ie} e ( {N}_{ie}^{β} {S}_{i} ( {h}_{i} ) + {p}_{ie} ( t ) ) , \qquad ( 4 )$$

$$\frac{{d}^{2} {I}_{ei} ( t )}{{dt}^{2}} + 2 {γ}_{ei} \frac{{dI}_{ei} ( t )}{dt} + {γ}_{ei}^{2} {I}_{ei} ( t ) = {Γ}_{e} {γ}_{ei} e ( {N}_{ei}^{β} {S}_{e} ( {h}_{e} ) + {p}_{ei} ( t ) ) , \qquad ( 5 )$$

$$\frac{{d}^{2} {I}_{ii} ( t )}{{dt}^{2}} + 2 {γ}_{ii} \frac{{dI}_{ii} ( t )}{dt} + {γ}_{ii}^{2} {I}_{ii} ( t ) = {Γ}_{i} {γ}_{ii} e ( {N}_{ii}^{β} {S}_{i} ( {h}_{i} ) + {p}_{ii} ( t ) ) , \qquad ( 6 )$$

$$where$$

$${S}_{j} ( {h}_{j} ) = \frac{{S}_{j}^{max}}{( 1 + exp ( - \sqrt{2} \frac{( {h}_{j} - \tilde{{μ}_{j}}}{{σ}_{j}} ) )} ; j = e , i . \qquad ( 7 )$$

These equations describe the interactions between inhibitory and excitatory neuronal populations. Specifically, the temporal dynamics of mean soma membrane potentials for the excitatory (hₑ(t)) and inhibitory (hᵢ(t)) populations are described in Equations (1) and (2). The temporal dynamics of the synaptic activity, Iₑₑ(t), Iᵢₑ(t), Iₑᵢ(t), and Iᵢᵢ(t), are given by Equations (3)-(6). The relationship between the mean population firing rate, Sⱼ, and the mean soma membrane potentials of the respective population is given in Equation (7). Prior research has shown that the local field potential measured in the EEG is linearly proportional to the mean soma membrane potentials of the excitatory populations, hₑ(t).

The neural population model data also includes one or more neural model parameters. Table 1 shows the model parameters for the above described neural population model, along with their corresponding physiological ranges.

TABLE 1
Physiological parameters of the model presented along with their physiological ranges.

| No | Physiological parameter | Symbol | Min | | Max | |
|---|---|---|---|---|---|---|
| 1 | Mean resting membrane potential of the excitatory population | hₑ^{rest} | −80 | mV | −60 | mV |
| 2 | Mean resting membrane potential of the inhibitory population | hᵢ^{rest} | −80 | mV | −60 | mV |
| 3 | Mean Nernst membrane potential of the excitatory population | hₑ^{eq} | −20 | mV | 10 | mV |
| 4 | Mean Nernst membrane potential of the inhibitory population | hᵢ^{eq} | −20 | mV | 10 | mV |
| 5 | Maximum mean firing rate of the excitatory population | Sₑ^{max} | 0.05/ms | | 0.5/ms | |
| 6 | Maximum mean firing rate of the inhibitory population | Sᵢ^{max} | 0.05/ms | | 0.5/ms | |
| 7 | Firing thresholds of the excitatory population | μ̅ₑ | −55 | mV | −40 | mV |
| 8 | Firing thresholds of the inhibitory population | μ̅ᵢ | −55 | mV | −40 | mV |
| 9 | Std. deviation of firing thresholds of the excitatory population | σₑ | 2 | mV | 7 | mV |
| 10 | Std. deviation of firing thresholds of the inhibitory population | σᵢ | 2 | mV | 7 | mV |
| 11 | Passive membrane decay time const. of the excitatory population | τₑ | 5 | ms | 150 | ms |
| 12 | Passive membrane decay time const. of the inhibitory population | τᵢ | 5 | ms | 150 | ms |
| 13, 14 | Excitatory postsynaptic potential rate constant | γₑₑ, γₑᵢ | 1.0/ms | | 1.0/ms | |
| 15, 16 | inhibitory postsynaptic potential rate constant | γᵢₑ, γᵢᵢ | 0.01/ms | | 0.5/ms | |
| 17 | Postsynaptic potential amplitude of the excitatory population | Γₑ | 0.1 | mV | 0.1 | mV |
| 18 | Postsynaptic potential amplitude of the inhibitory population | Γᵢ | 0.1 | mV | 0.1 | mV |

| 19 | Rate of the excitatory input to the excitatory population | pₑₑ | 0.0/ms | 10.0/ms |
|---|---|---|---|---|
| 20 | Rate of the excitatory input to the inhibitory population | pₑᵢ | 0.0/ms | 10.0/ms |
| 21 | Rate of the inhibitory input to the excitatory population | pᵢₑ | 0.0/ms(fixed) | |
| 22 | Rate of the inhibitory input to the inhibitory population | pᵢᵢ | 0.0/ms(fixed) | |
| 23 | Total number of connections an excitatory neuron receives from | Nₑₑ^{β} | 2000 | 5000 |
| | nearby excitatory neurons | | | |
| 24 | Total number of connections an inhibitory matron receives from | Nᵢₑ^{β} | 100 | 1000 |
| | nearby excitatory neurons | | | |
| 25 | Total number of cminections an excitatory neuron receives from | Nₑᵢ^{β} | 2000 | 5000 |
| | nearby inhibitory neurons | | | |
| 26 | Total number of connections an inhibitory neuron receives from | Nᵢᵢ^{β} | 100 | 1000 |
| | nearby inhibitory neurons | | | |

In some embodiments, the data handler 108 is configured to process the selected EEG data and/or the neural population model data (i.e. at step 305) prior to the generation of the fitted model data. For example, in the described embodiments the data handler 108 is configured to produce a spectral frequency domain representation of each time domain signal in the EEG data. Welch's method of averaging (as described in Welch P. The use of fast Fourier transform for the estimation of power spectra: a method based on time averaging over short, modified periodograms. IEEE Transactions on audio and electroacoustics. 1967; 15(2):70-73) the spectra derived from multiple overlapping time segments is used to estimate the single spectrum for a particular individual. This approach improves the precision of the power spectral density (PSD) estimate by sacrificing some spectral resolution. The data handler 108 performs spectral estimation using sampling frequencies and window functions as selected during configuration. Although model parameter fitting is performed based on spectral representations of the EEG data for the described embodiments, in other embodiments time domain EEG signals are used to perform the fitting. In some embodiments, the data handler 108 is configured to process the neural model population data, such as to perform parameter simplification operations prior to model data fitting. For example, by equating one or more of the parameters (as described below), the effective dimensionality of the model is reduced, improving computational efficiency.

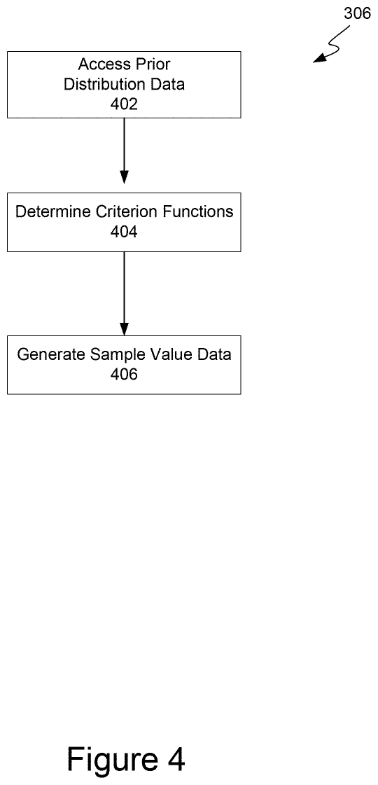

At step 306, a model fitting process is executed to generate fitted model parameter data representing a set of estimated values for the parameters of the neural population model. FIG. 4 is a flow diagram of a model fitting process of the described embodiments as performed by the sampling and estimation unit 110, which involves accessing prior distribution data representing an assumed distribution for each parameter (i.e. at step 402). At step 404, the sampling and estimation unit 110 determines estimation criterion functions to be used in the fitting procedure (i.e. to assess the candidate values based, at least in part, on the EEG data). In the described embodiments, the estimation criterion functions include: i) a predicted model spectrum function; and ii) a corresponding likelihood function. These functions are used to produce spectral and likelihood values for candidate model parameters that are considered during fitting (i.e. such that the best parameter estimates are derived based on the EEG spectrum data and the estimation criterion functions).

The sampling and estimation unit 110 determines the predicted model spectrum and likelihood functions based on the neural population model. For the default neural model of the described embodiments, the predicted model spectrum function is calculated from the spatially homogeneous version of the full model equations (i.e. Equations (1)-(6) above). In the described implementation, the predicted model spectrum is calculated subject to the following assumptions:
i) The system is linearly stable in the vicinity of particular solutions (e.g., those corresponding to the resting eyes-open and eyes-closed spectra in the evaluations discussed below).
ii) The excitatory rate constants γₑₑ and γₑᵢ are equal, as are the inhibitory rate constants γᵢₑ and γᵢᵢ.
iii) The measured EEG signal is proportional to the excitatory mean soma membrane potential, hₑ.
iv) The linearised system is driven by Gaussian white noise fluctuations on the external excitatory to excitatory signal pₑₑ.

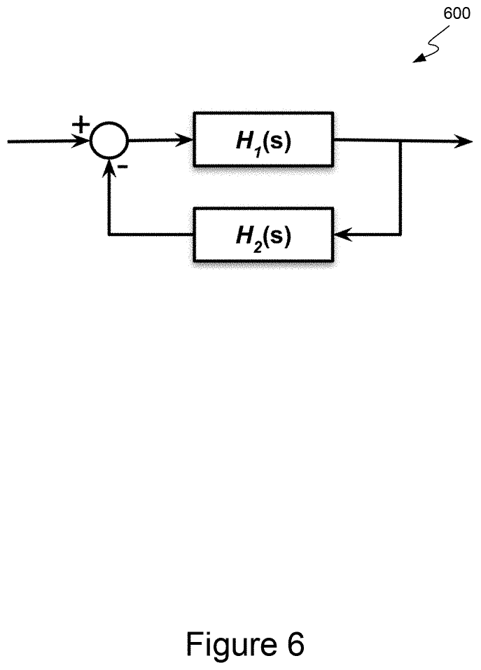

Under these assumptions, it can be shown that the linear system transfer function, T(s), is that of a simple feedback system 600, as shown in FIG. 6, involving two third order filters H₁(s) and H₂(s) according to:

$$T ( s ) = \frac{{H}_{1} ( s )}{1 + {H}_{1} ( s ) {H}_{2} ( s )} , \qquad ( 8 )$$

$${H}_{1} ( s ) = \frac{{k}_{13}}{{k}_{13} {k}_{31} - {k}_{11} ( s ) {k}_{33} ( s )} , \qquad ( 9 )$$

$${H}_{2} ( s ) = \frac{{k}_{15} {k}_{24} {k}_{41} {k}_{52}}{{k}_{13} \{ {k}_{22} ( s ) {k}_{55} ( s ) - {k}_{26} {k}_{62} \}} . \qquad ( 10 )$$

The polynomials k₁₁(s) and k₂₂(s) are linear in s, and k₃₃(s) and kₛₛ(s) are quadratic in s. Let θ be a vector of model parameters. Given that the spectra are assumed to arise from a white noise spectrum filtered by this transfer function, the expected value of the spectral estimate at frequency w, given θ, has the form:

$$〈 S ( ω ) 〉 = α \hat{S} ( ω \vert θ ) = α {\vert \frac{{H}_{1} ( i ω )}{1 + {H}_{1} ( i ω ) {H}_{2} ( i ω )} \vert}^{2} , \qquad ( 11 )$$

where the constant α accounts for the unknown driving amplitude and for attenuation due to volume conduction and other (frequency-independent) effects. The value for α is found using a least-squares fit to the measured spectral estimates. The analytic result is:

$$α = \frac{{\sum }_{n} {S}_{n} {\hat{S}}_{n} ( θ )}{{\sum }_{n} {\hat{S}}_{n}^{2} ( θ )} , where {S}_{n} ≡ S ( {ω}_{n} ) ; n = 1 , … , N . \qquad ( 12 )$$

The sampling and estimation unit 110 determines the likelihood function for the above described predicted spectrum function by ignoring the effects of window overlap and non-uniform window shape on the resulting distributions. Furthermore, assuming sufficiently high sampling rates and negligible measurement noise, the spectral estimate from the Welch periodogram at each sampled frequency ωₙ, =2πfₙ; n=1, 2, . . . , N is approximated as an independent random variable with a known distribution. These simplifications are made to improve the computational requirements of the function determination step 404. However, in other embodiments the aforementioned effects may be taken into account.

With these simplifications, the probability distribution function (pdf) for the spectral estimate, Sₙ, is a gamma distribution:

$$f ( {S}_{n} ; K , Θ ) = \frac{{S}_{n}^{K - 1} {e}^{- \frac{{S}_{n}}{{θ}_{n}}}}{{Θ}_{n}^{K} Γ ( K )} ; {S}_{n} > 0. \qquad ( 13 )$$

For non-zero frequencies, the shape parameter K is found from the number of epochs averaged in the periodogram (for zero frequency, replace variable K with K/2). The scale parameter is given by:

$${Θ}_{n} ( θ , α ) = α \frac{{\hat{S}}_{n} ( θ )}{K} , where {\hat{S}}_{n} ( θ ) ≡ \hat{S} ( {ω}_{n} \vert θ ) ; n = 1 , … , N . \qquad ( 14 )$$

The likelihood function for the vector of spectral estimates, S=[S₁S₂ . . . S_{N}]^{T}, given parameter values θ, is then the product of the distributions of the individual spectral estimates:

$$p ( S \vert θ , α ) = {( \frac{1}{Γ ( K )} )}^{N} ( {\prod }_{n} \frac{{( \frac{{S}_{n}}{{\hat{S}}_{n} ( θ )} )}^{K - 1}}{{\hat{S}}_{n} ( θ )} ) {( \frac{K}{α} )}^{NK} exp ( - \frac{K}{α} {\sum }_{n} \frac{{S}_{n}}{{\hat{S}}_{n} ( θ )} ) . \qquad ( 15 )$$

The constant α is adjusted to give the maximum likelihood fit of the model spectrum to the target experimental spectrum. The analytic result is that:

$$α = \frac{1}{N} {\sum }_{{n = 1}_{}}^{N} \frac{{S}_{n}}{{\hat{S}}_{n} ( θ )} . \qquad ( 16 )$$

The likelihood function, as based on the model parameters, is then:

$$p ( S \vert θ ) = {( \frac{{K}^{K} {e}^{- K}}{Γ ( K )} )}^{N} {( \frac{1}{\frac{1}{N} {\sum }_{n = 1}^{N} \frac{{S}_{n}}{{\hat{S}}_{n} ( θ )}} )}^{NK} ( {\prod }_{n} \frac{{( \frac{{S}_{n}}{{\hat{S}}_{n} ( θ )} )}^{K - 1}}{{\hat{S}}_{n} ( θ )} ) . \qquad ( 17 )$$

At step 406, the sampling and estimation unit 110 generates sample data representing estimated model parameter values according to a fitting process and the estimation criterion functions. The fitting process may be selected during configuration, or determined by the controller 106 according to a default setting of the NAC 102. Specifically, the described component implements two processes for fitting the model parameters to the spectral frequency EEG data, namely particle swarm optimisation (PSO) based on least squares minimisation, and a Markov Chain Monte Carlo (MCMC) method with maximum likelihood estimation.

As known by those skilled in the art, PSO is an optimization algorithm inspired by swarming behaviour in nature to process knowledge in the course of searching for the best solution in a high-dimensional parameter space [symbol] ^{D}. At the individual level, a particular particle p in a particular iteration represents a distinct candidate solution Xₚ ∈ [symbol] ^{D} whose quality is defined by a cost function. Throughout successive iterations, the particle moves around in the search-space in the direction and velocity guided by its local best known position Lₚ ∈ [symbol] ^{D} as well as the global best known position G∈ [symbol] ^{D} in such a way that for any iteration p's velocity is given by:

$${V}_{p} ← ψ {V}_{p} + {ϕ}_{ind} \cdot ( {L}_{p} - {X}_{p} ) + {ϕ}_{soc} \cdot ( G - {X}_{p} ) \qquad ( 18 )$$

where ψ is a predefined inertia weight, as proposed by Shi and Eberhart (see A modified particle swarm optimizer. In: Evolutionary Computation Proceedings, 1998. IEEE World Congress on Computational Intelligence., The 1998 IEEE International Conference on. IEEE; 1998. p. 69-73), while ϕ_{ind}=rand (0,ϕ_{ind}^{max}) and ϕ_{soc}=rand(0,ϕ_{soc}^{max}) are weights given to individual versus social interaction, respectively (here rand(0,c^{max}) represents a random value between 0 and c^{max}). The algorithm iteratively updates the global best known position G until a stopping criterion is reached.

When utilising the PSO fitting process, the sampling and estimation unit 110 performs parameter estimation by finding the vector of parameters \hat{θ} that minimizes a least squares (LS) cost function C given by the sum of squared residuals between the measured spectrum S and the normalized (predicted) model spectrum αŜ(θ):

$$C = {\sum }_{n} {( α {\hat{S}}_{n} ( θ ) - {S}_{n} )}^{2} . \qquad ( 19 )$$

An initial sample generation process is performed involving using 1000 independent instantiations of the PSO to find 1000 different parameter estimates. By default, the algorithm uses 80 particles for each swarm. Parameter starting points are chosen by random sampling from a uniform distribution over the physiologically-relevant ranges, as given in Table 1. During the parameter search, each parameter is forced to stay within its physiologically-relevant range by assigning a high cost to particles with values outside these ranges. This is similar to employing a Bayesian prior that is flat over the acceptable parameter range, and zero elsewhere. This results in a preliminary set of 1000 parameter estimates \hat{θ}.

The samples produced from the initial sampling process described above are then filtered to remove samples that are not acceptable fits. The sampling and estimation unit 110 is configured to only accept the 10 percent of estimates that have the lowest cost functions. In other embodiments, alternative filtering strategies may be implemented.

Having selected samples of \hat{θ}, the sampling and estimation unit 110 generates the marginal posterior distributions of the parameters involving the construction of kernel density estimates.

Alternatively, the sampling and estimation unit 110 can use a MCMC method to perform the fitting operation. Under this process, an explicitly Bayesian framework is employed by treating the parameters as random variables in order to integrate the parameter estimation and posterior distribution determination steps While PSO involves both sample generation and sample selection steps, MCMC involves performing long sequence sample generation based on log likelihood ratio (with a burn in) and resampling, when appropriate, to generate averages (for KLD estimation). Given a known prior distribution, p₀(θ), it is desired to find the posterior distribution conditioned on the observed spectrum S is given by:

$$p ( θ \vert S ) = \frac{p ( S \vert θ ) {p}_{0} ( θ )}{p ( S )} . \qquad ( 20 )$$

In the described embodiments, the calculation is simplified by searching for a local maximum in the vicinity of the MCMC sampled parameter that maximizes the likelihood function (as given by Equation (17)) evaluated for the observed spectrum. The sampling and estimation unit 110 performs Monte Carlo estimation by drawing values randomly from the known prior distribution. To obtain these values, the Metropolis-Hastings Markov chain Monte Carlo (MCMC) algorithm is implemented based on the log likelihood ratio that follows from the likelihood function described above, as follows:

$$ln ( \frac{p ( S \vert {θ}_{n + 1} )}{p ( S \vert {θ}_{n} )} \frac{{p}_{0} ( {θ}_{n + 1} )}{{p}_{0} ( {θ}_{n} )} ) = KN \{ ln ( \frac{1}{N} {\sum }_{m} \frac{{S}_{m}}{{\hat{S}}_{m} ( {θ}_{n} )} ) - ln ( \frac{1}{N} {\sum }_{m} \frac{{S}_{m}}{{\hat{S}}_{m} ( {θ}_{n + 1} )} ) \} + K {\sum }_{m = 1}^{N} \{ ln {\hat{( S}}_{m} ( {θ}_{n} ) ) - ln ( {\hat{S}}_{m} ( {θ}_{n + 1} ) ) \} + ln ( {p}_{0} ( {θ}_{n + 1} ) ) - ln ( {p}_{0} ( {θ}_{n} ) ) \qquad ( 21 )$$

The sampled sets obtained for each target spectrum consist of 10⁶ MCMC samples with the step size (of normalised parameter values) adjusted during a burn-in phase of length 40000 to yield an acceptance ratio in the vicinity of 0.25. When appropriate, the long sampled sequence is resampled, typically to yield sequences of length 1000 upon which to base averages. For consistency with the particle swarm approach, the prior distribution is assumed to be uniform over its support.

The parameter analyser 112 receives data representing the best-fit model parameters \hat{θ} and corresponding posterior distribution (as determined in step 306). Returning to the flow diagram of FIG. 3, given the fitted model parameters and corresponding posterior distribution produced as a result of the model fitting procedure, at step 308 the NAC 102 generates parameter identifiability data representing measures of model parameter identifiability.

In the described embodiments, identifiability measures are generated for each model parameter individually (referred to as “individual identifiability”). In some embodiments, identifiability is also assessed for one or more combinations of particular model parameters resulting in the generation of “collective” identifiability measures (as described below).

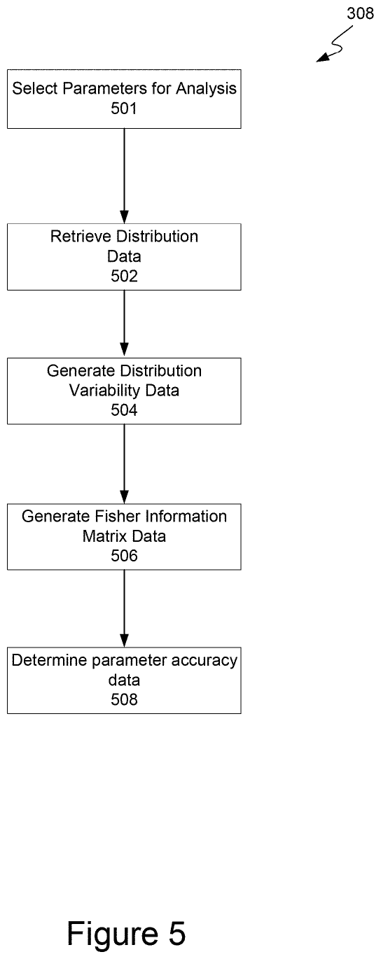

FIG. 5 is a flow diagram showing the generation of data representing measures of individual parameter identifiability. At step 501, particular model parameters are selected for identifiability assessment. The selected parameters may be specified by the user 103 (such as during configuration), or automatically selected by the NAC 102 (such as in accordance with a default option).

At step 502, the parameter analyser 112 retrieves (via the controller 106) distribution data for each selected parameter. At step 504, distribution variability data is generated to represent the individual identifiability of each selected parameter. In the described embodiments, the Kullback-Leibler divergence (KLD) is calculated to measure the information gained for a single parameter as a result of the measurement of the spectrum. The parameter analyser 112 calculates the KLD to measure the change in the marginal posterior distribution of each selected parameter relative to its marginal prior:

$${D}_{KL}^{( i )} ( S ) = \int {p}_{i} ( {θ}_{i} \vert S ) ln \frac{{p}_{i} ( {θ}_{i} \vert S )}{{p}_{0} ( {θ}_{i} )} d {θ}_{i} . \qquad ( 22 )$$

When the KLD is used to measure how much posterior parameter estimates differ from the predicate uniform priors for parameter fitting performed with MCMC samples, the integral is numerically evaluated using marginal distributions approximated by kernel density estimates using 1000 parameter values resampled from the full MCMC sampled parameter set for the given spectrum. In this implementation, the prior distributions are assumed to be uniform over their support. For consistency, the posterior kernel estimates are truncated to have the same support. The kernel density estimate for a given parameter is sampled at 100 points over its support and the integral estimated numerically. For parameter fitting performed with PSO, due to the limited number of independent samples, the integral is estimated using a 10 bin histogram approximation.

In some embodiments, the parameter analyser 112 is configured to generate parameter identifiability data that also represents the identifiability of a combination of model parameters (i.e. referred to as measures of “collective” identifiability) and/or the sloppiness of the model. At (optional) step 506, collective parameter identifiability is determined based on the Fisher Information Matrix (FIM) of the model. Eigenvalues of the Fisher information matrix for the distribution P(s|θ) are given by:

$${I}_{μ v} ( θ ) = \int p ( S \vert θ ) \frac{\partial ln P ( S \vert θ )}{\partial {θ}^{μ}} \frac{\partial ln P ( S \vert θ )}{\partial {θ}^{v}} {d}^{N} S \qquad ( 23 )$$

The parameter analyser 112 evaluates Equation (23) resulting in an expression involving only the derivatives of the model spectral estimates at the desired parameter values:

$${I}_{μ v} ( θ ) = K {\sum }_{n}^{ } \frac{\partial ln {\hat{S}}_{n} ( θ )}{\partial {θ}_{μ}} \frac{\partial ln {\hat{S}}_{n} ( θ )}{\partial {θ}_{v}} \qquad ( 24 )$$

In the described embodiments, the parameter analyser 112 is configured to evaluate Equation (24) using a 5-point finite difference approximation, where the resulting products are summed over the sampled frequencies. Numerical experiments with surrogate matrices suggest that the eigenvalues calculated may be reliable over some 10 orders of magnitude. In the described implementation, the parameter analyser 112 assumes the FIM to be positive semidefinite and of less than full rank for the modelled spectra. Negative eigenvalues and eigenvalues smaller than 10⁻¹⁰ times the largest eigenvalue, and are therefore taken to be zero.

The parameter identifiability data produced by analyser 112 includes the distribution variability data, and the Fisher Information Matrix data if generated. In some embodiments, the parameter identifiability data also includes parameter accuracy data representing one or more measures of the accuracy and/or precision of the best fit values.

Referring to the flow diagram of FIG. 3, the parameter identifiability data is processed to generate a set of determinative model parameters, these being the parameters that characterise the features of the EEG data. In some embodiments, the determinative parameter set is generated automatically by the parameter analyser 112 based on identifiability criteria that may be configured by the user 103 (such as, for example, N-best selection to determine the identifiable parameters as those with the N highest KLD values).

In other embodiments, the NAC 102 is configured to accept user input to generate the determinative parameter set. For example, the parameter identifiability data may be displayed to the user 103 allowing the user 103 to select particular parameters to form the determinative set (e.g., based on the user's own consideration of the KLD and/or FIM values).

The parameter analyser 112 can be configured to generate a measure of the model sloppiness based on the parameter identifiability data. Model sloppiness is defined by the existence of eigenvalues of the Fisher information matrix (FIM) being roughly uniformly spaced over a log scale. The eigenvalues of the FIM identify the sloppy and stiff parameter eigenvector directions. The larger FIM eigenvalues define eigenvector directions corresponding to particular identifiable parameter combinations. To understand the parameters that contribute the most to each (identifiable) eigenvector the angular distance between each parameter direction and a given eigenvector is computed. The closer the angular distance to 0 degrees or 180 degrees, the greater the parameter contributes to the parameter combination and thus the more identifiable that parameter. In some embodiments, analysis of simulated spectral data may also be used to assess the accuracy and/or precision of parameters for the purpose of composing the determinative parameter set.

The generated determinative parameter data is accessed by the controller 106, and is subsequently made available to the user 103 (i.e., via UI 104). The user 103 can then review the determined parameters in view of the EEG data and its characteristics of interest, and, in some embodiments, provide the NAC 102 with instructions to simplify the neural population model. In response to such instructions, the determinative parameter data is processed to generate optimised neural population model data representing a new neural population model with a reduction in the degrees of freedom (relative to the initial model). Operations performed by the parameter analyser to generate the (new) optimised neural population model may include grouping together, eliminating, or averaging subsets of parameters.

At step 310, the NAC 102 is configured to utilise the determinative parameter data to determine aspects of brain function (or brain state activity) of a particular subject (e.g., one of individuals 131-13M, or another individual whose EEG data has not been received in step 302 to identify the determinative parameters). As illustrated by FIG. 3b, the process of assessing the brain function of the subject involves receiving EEG data representing an EEG recording obtained from the subject (i.e., at step 312). The EEG data of the subject is received in the form of a time domain signal that is, at step 314, processed to produce frequency spectrum representations according to the processes described herein above (i.e., for step 305). Segmentation is performed to the subject EEG data such that spectral analysis is performed for over-lapping time domain data segments of a fixed maximum length (i.e., in order to validate assumptions of signal stationarity).

At step 316, model parameter data is generated by fitting the neural population model to each EEG spectrum (as generated at step 314). Model fitting proceeds according to a fitting process, which may selected as, for example, the PSO or MCMC processes described herein above (i.e., at step 306). The fitted model parameter data includes representations of the estimated values for, at least, each determinative model parameter (referred to as the “determinative parameter estimates”).

In some embodiments, the determinative parameter estimates are processed, at step 318, to apply particular adjustment or transformation operations to the values. For example, the values of a particular determinative parameter may be scaled to ensure that each value falls within a scaling range. The scaling range may be determined experimentally by applying the neural population model fitting process to data collected from one or more validation individuals. Alternatively, the NAC 102 can be configured to apply predetermined scaling factors to each parameter value, where the scaling factors may be constant (i.e. “global”) in nature, or may vary dynamically based on the characteristics of the subject (e.g., as specified by the user 103).

An indication of brain activity is determined for the subject based on the processed determinative parameter estimates. Since the determinative parameters are characteristic of particular EEG feature(s), the scaled (or otherwise processed) estimates of these parameters provide an indication of an underlying aspect of brain function that is known to be associated with the particular EEG feature(s). The brain activity indication output by the NAC 102 can be subsequently used in a clinical application, such as for example to determine whether or not to perform a particular operation (i.e., at step 322) without requiring a direct analysis of the subject's EEG data.

The indication of brain activity produced by the NAC 102 can be a measurement in the form of a single value (e.g., as obtained from aggregating a fixed number of estimated values for each determinative parameter in the case of an isolated EEG recording of the subject). Alternatively, the indication of brain activity can be a sequence of determinative parameter estimates obtained from a continuously recorded EEG signal. This provides a clinician with corresponding time sequential measures of brain activity that can be utilised to monitor the brain function of the subject (i.e. to measure it at consecutive time instants) in response to operations of a procedure.

As shown in FIG. 3b, the indication of brain activity output by the NAC 102 can be utilised as part of a closed-loop feedback system. Specifically, an assessment of brain function based on determinative parameter estimates produced by the subject's real-time EEG recordings can be used to determine whether to perform a particular clinical operation to the subject (such as at step 322).

### Model Parameter Estimation for Alpha-Rhythmic EEG Data

The neurophysical assessment processes described herein above were applied to EEG data containing alpha-rhythmic features for the purpose of: i) generating a determinative parameter set for the above described neural population model to characterise the alpha-rhythm (where this step is referred to as an “evaluation” of the model); and ii) subsequently utilising estimates of the determinative parameters to provide a measure of brain function (or “activity”) during surgical procedures.

The evaluation proceeded using experimental EEG data representing EEG signals collected from 82 individuals in a resting state. The experimental EEG data received by the NAC 102 to perform the evaluation is a subset of a larger EEG evaluation dataset, where the evaluation dataset consists of data obtained over 14 experimental tasks performed by 109 subjects. The EEG recordings were obtained via the use of 64 electrodes according to the International 10-10 System. The full EEG evaluation dataset, collected and contributed by Schalk et al. (i.e. in Schalk G, McFarland D J, Hinterberger T, Birbaumer N, Wolpaw J R. BCI2000: a general-purpose brain-computer interface (BCI) system. IEEE Transactions on biomedical engineering. 2004; 51(6):1034-1043) using the BCI2000 instrumentation system, is available for public access in PhysioNet (https://www.physionet.org/pn4/eegmmidb/). The EEG recordings used for the evaluation included signals from the Oz electrode, and from individuals whose EEG spectrum exhibited an alpha rhythmic feature (i.e. in the form of clear alpha peaks as obtained during the associated eyes-closed task). It is noted that the alpha band activity obtained from a single electrode (as from the Oz electrode in the above evaluation) represents the superposition of multiple independent spatially distributed, alpha rhythm generators. It may be possible to differentiate between these different sources by jointly fitting the model to signals from multiple electrodes.

The NAC 102 was configured to use the default neural population model described herein above. Although the model prescribes 26 physiological parameters (see Table 1), for the purposes of the evaluation the data handler 108 was configured to perform model simplification by setting pᵢₑ and pᵢᵢ to fixed constants, and the postsynaptic potential rate as γₑₑ=γₑᵢ≡γₑ and γᵢₑ=γᵢᵢ≡γᵢ. The resulting neural model has 22 effective parameters where values were calculated by applying a fitting process to the experimental EEG data.

Processing of the EEG data involved generating a one-minute EEG signal associated with a particular individual, sampled at 160 Hz, which was divided into segments using a 4-second Hamming window with an overlap of 50%. The data handler 108 was configured to assume stationarity over the one-minute EEG signal, where stationarity means that it is the parameters that are constant, while the states are allowed to vary about a stable fixed point. Deviations of the state away from the fixed point were assumed to be small enough to allow linearization of the model.

Though parameters were assumed to be constant within a given EEG signal, they can of course vary between different recordings (and thus individuals). Because of well-known nonlinearities and non-stationarities in EEG recordings, the linearized model was used in inference procedures only for frequencies between 2 Hz and 20 Hz. It can be assumed that well over 95% of the spectral power in the resting M/EEG falls below 30 Hz. Indeed, typical estimates of resting M/EEG spectral edge frequency (SEF95) (i.e. the 139 frequency below which 95% of the spectral power is contained) are in the range of 24-26 Hz.

To demonstrate the accuracy of the inference methods, parameters can be estimated using a simulated spectrum, where the underlying parameter set is known (and referred to as the ground truth). The maximum likelihood estimate found for Subject 77 was used to choose a plausible parameter set for this test. The simulated spectrum was calculated by sampling each frequency channel from a gamma-distributed model prediction.

Particle swarm optimization (PSO) and Markov Chain Monte Carlo (MCMC) based parameter fitting processes were applied, as described above. Estimation of 22 posterior distributions was performed (i.e. for each unknown model parameter), using both algorithms, for each of the 82 different EEG spectra.

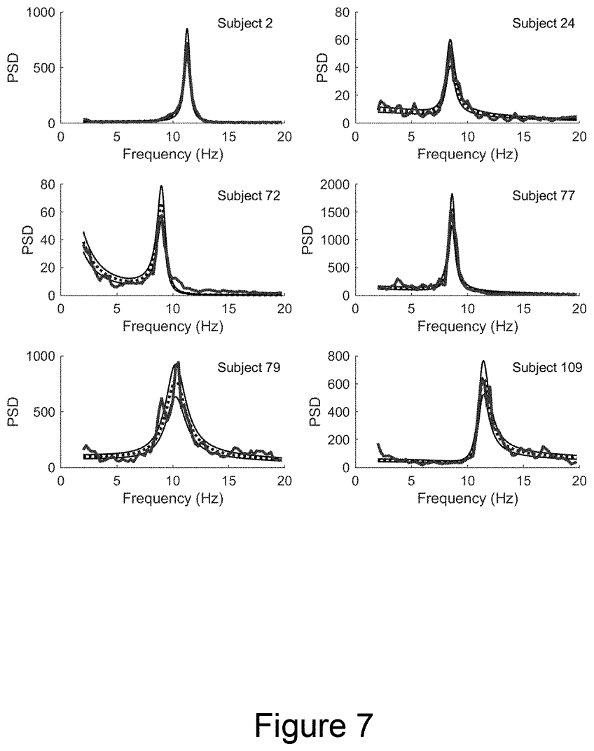

FIG. 7 is a set of graphs of power spectra density as a function of frequency for six subjects illustrating the use of PSO to find best parameter fits using least squares (LS). Each graph shows a comparison of the model spectra (blue dotted line) fit to the experimental spectra (red thick line) by least squares (LS) minimization using particle swarm optimization, for a select set of subjects. Also shown are the 16% and 84% quantiles based on the gamma distribution for the fitted spectra (thin black lines). The subjects have been selected to show the range of spectra included in the full data set.

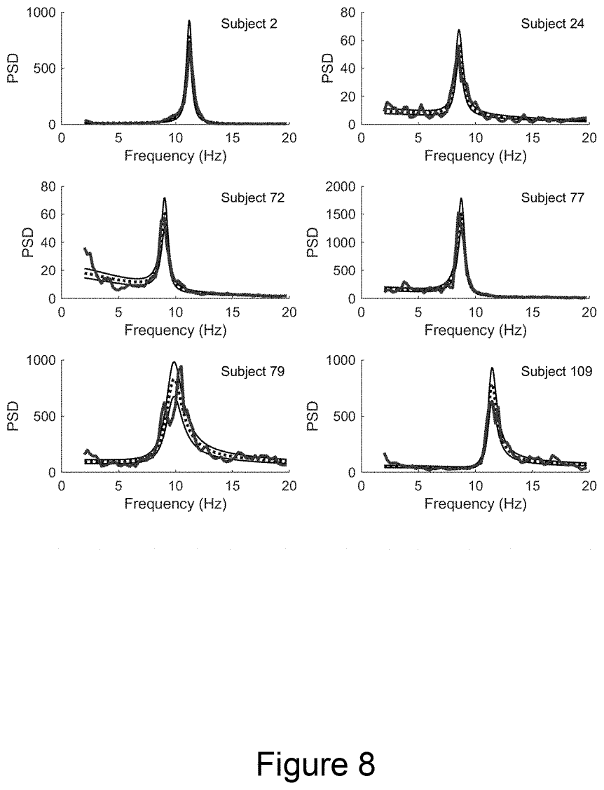

FIG. 8 is a set of graphs of power spectra density as a function of frequency for six subjects illustrating the use of a MCMC method to find best parameter fits, which samples solutions based on maximum likelihood (ML) estimations. The figure shows a comparison of model spectra (blue dotted line) fit to experimental spectra (red thick line) by maximum likelihood (ML) estimation using MCMC. Also shown are the 16% and 84% quantiles (thin black lines). The subjects are the same as in FIG. 7. It should be noted that LS and ML fits are expected to differ in this case, since the LS fits are more sensitive to deviations in the unweighted spectral power (typically in regions with larger power) whereas the ML fits are more sensitive to deviations in regions where the variance of spectral power is small (typically in regions of lower spectral power).

Although the fits depicted in FIGS. 7 and 8 have similarities, subtle differences can be observed. For example, in subject 72 the ML fit performs better on regions with lower power, but less well in regions with higher power. This difference is expected as, while LS are computed over unweighted power spectra, ML favors frequencies with lower variances which typically are those with lower power spectra.

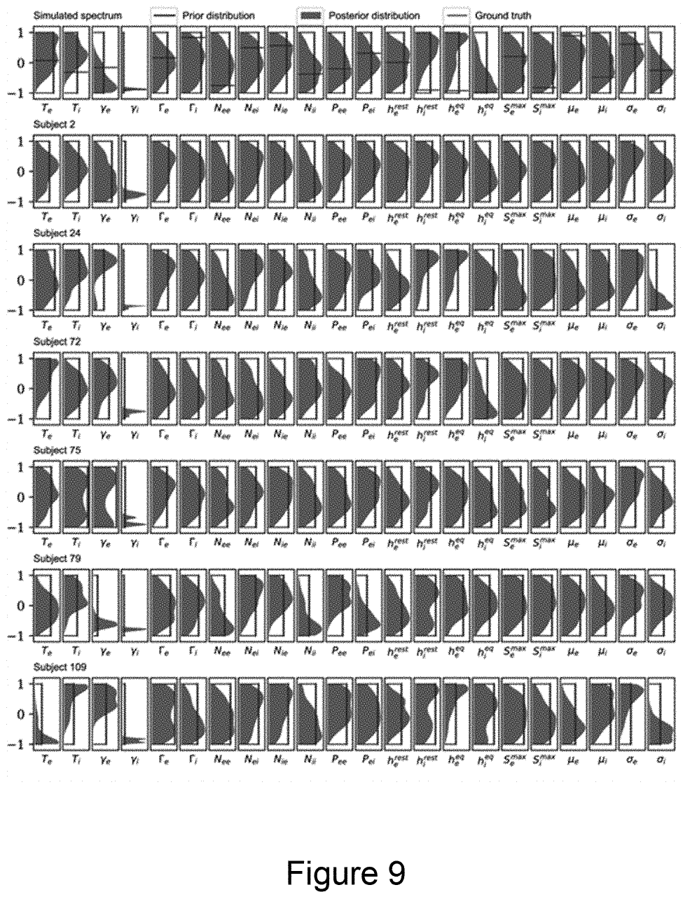

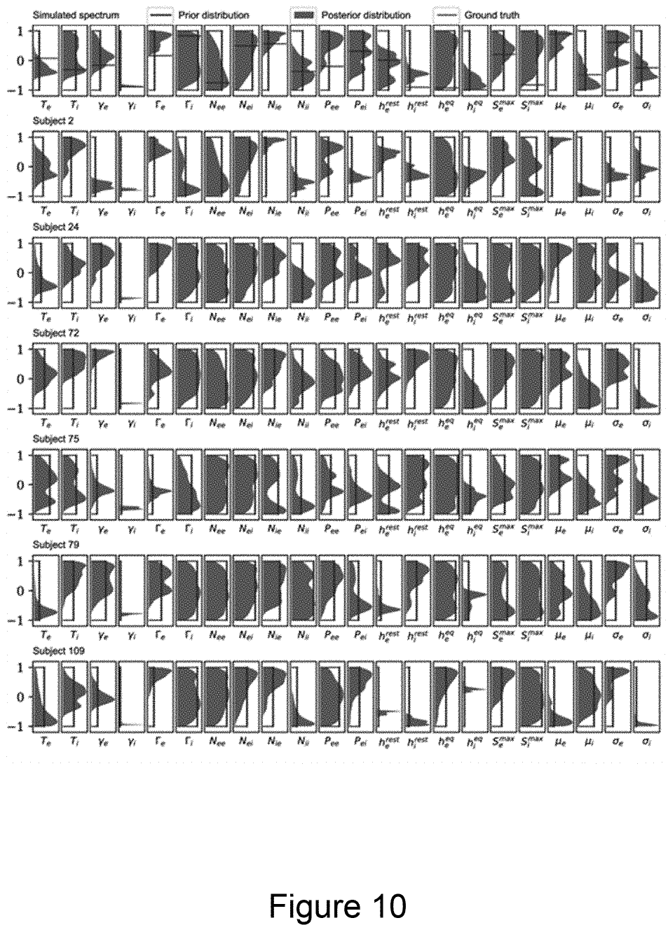

FIGS. 9 and 10 depict sets of graphs showing, for PSO and MCMC sampling respectively, a comparison of the posterior marginal distribution (solid color) and prior marginal (green line) distributions for the selected subjects. The top row, which corresponds to analysis of the simulated spectrum, also shows the ground truth value (red). Each parameter is plotted in normalized coordinates, where −1 corresponds to the lower limit of the plausible parameter interval and +1 corresponds to the upper limit (see Table 1).

When using PSO, the distributions are based on kernel density estimates from the best 100 of 1000 randomly seeded particle swarm optimizations for each subject. The seeds are uniformly distributed over the allowed parameter ranges. It is observed that, across the full set of 82 subjects, only the parameter γᵢ is significantly constrained. All other parameters have nearly the same uncertainties as the prior.

When using MCMC, each distribution is based on a kernel density estimate from 1000 samples (sub-sampled from 10⁶ MCMC samples). As consistent with the observations from using PSO sampling, only parameter γᵢ is consistently constrained by the data when viewed across all subjects.

The posterior distributions found using PSO sampling are generally broader than those found using MCMC sampling. This behaviour is expected from the differences between the sampling methods: while MCMC sampling can retain correlations between samples even with significant subsampling, the different PSO samples are independent from one another. This demonstrates the superiority of the PSO approach, at least under the sampling conditions employed here. Nevertheless, both methods show that it is the postsynaptic potential rate constant of the inhibitory population, γᵢ, which is consistently constrained by the data across different subjects.

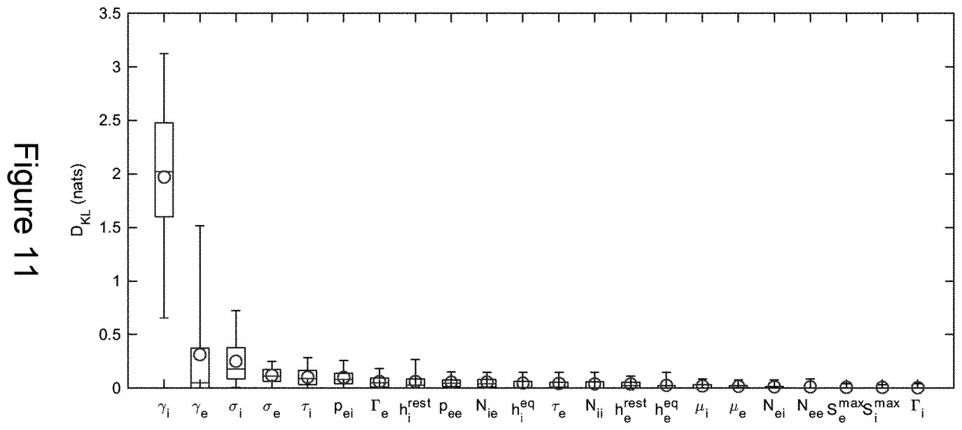

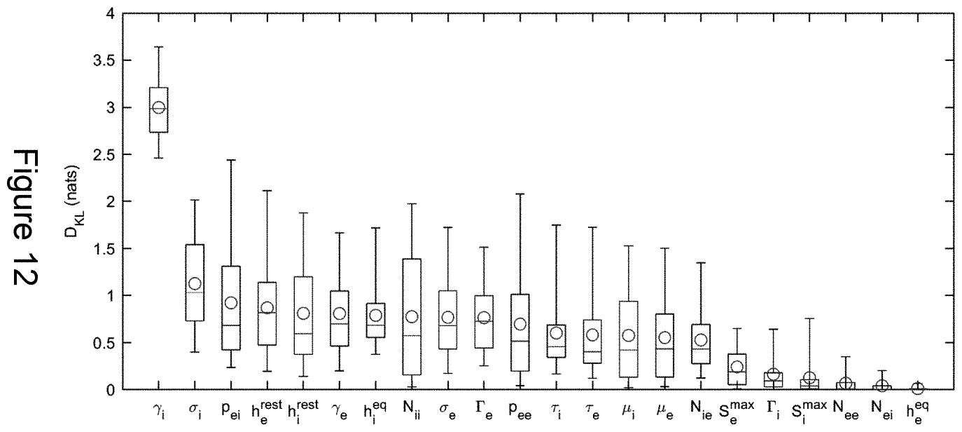

To evaluate the extent to which real data reduces the uncertainty in the estimation of each parameter, Kullback-Leibler divergence (KLD) values for each parameter, and from all 82 subjects, are calculated as shown in FIG. 11 (for PSO) and FIG. 12 (for MCMC). Specifically, FIG. 5 shows the KLD of marginal posterior parameter distributions calculated relative to uniform prior distributions. The posteriors are based on the best 100 of 1000 randomly seeded runs of particle swarm optimization (see FIG. 9). The boxes represent the 25% and 75% quartiles; the whiskers represent the 5% and 95% quantiles; the red lines show the median KLDs and the circles the mean KLDs of the distributions of the KLDs over the full set of 82 subjects.

The results confirm that parameter γᵢ is best-constrained by the data. Most other posterior distributions are only slightly narrower than their prior distribution. Table 2 shows an analysis of a simulated spectrum as performed to evaluate the accuracy and precision of the parameter estimates. Shown in the table is the mean of the deviation of the estimate from its ground truth value, normalized to the prior of the parameter. Also shown is the precision, given by the standard deviation of the sample estimates. The values in bold are the top three lowest values for each category. For both PSO and MCMC sampling, the estimate of γᵢ always has the highest precision, and also has one of the highest accuracies.

TABLE 2
Accuracy and precision of parameter estimates in
the analysis of the simulated spectrum.

| | PSO samples | | MCMC samples | |
|---|---|---|---|---|
| | Normalized | | Normalized | |
| Parameter | error | SD | error | SD |
| hₑ^{rest} | 0.0224 | 0.4771 | 0.1198 | 0.3772 |
| hᵢ^{rest} | 0.6165 | 0.4785 | 0.2231 | 0.1669 |
| hₑ^{eq} | 0.6402 | 0.5702 | 0.5069 | 0.5779 |
| hᵢ^{eq} | 0.2550 | 0.4864 | 0.1516 | 0.2082 |
| Sₑ^{max} | 0.1382 | 0.5405 | 0.0220 | 0.3967 |
| Sᵢ^{max} | 0.3847 | 0.5322 | 0.5205 | 0.4957 |
| μₑ | 0.4217 | 0.5281 | 0.1260 | 0.2655 |
| μᵢ | 0.2423 | 0.5216 | 0.0613 | 0.3217 |
| σₑ | 0.2215 | 0.4528 | 0.0972 | 0.3991 |
| σᵢ | 0.0064 | 0.3669 | 0.0459 | 0.2440 |
| τₑ | 0.1043 | 0.4970 | 0.1640 | 0.2075 |
| τᵢ | 0.3050 | 0.3934 | 0.2444 | 0.4614 |
| γₑ | 0.1939 | 0.3761 | 0.1747 | 0.3269 |
| γᵢ | 0.0071 | 0.0346 | 0.0156 | 0.0215 |
| Γₑ | 0.0972 | 0.4558 | 0.2641 | 0.2167 |
| Γᵢ | 0.4104 | 0.5393 | 0.3805 | 0.5452 |
| pₑₑ | 0.0708 | 0.4707 | 0.3750 | 0.3335 |
| pₑᵢ | 0.1199 | 0.4443 | 0.0031 | 0.4528 |
| Nₑₑ | 0.3600 | 0.5229 | 0.2575 | 0.5362 |
| Nₑᵢ | 0.2240 | 0.5955 | 0.1608 | 0.5410 |
| Nᵢₑ | 0.2087 | 0.4796 | 0.0784 | 0.2057 |
| Nᵢᵢ | 0.1042 | 0.4789 | 0.0108 | 0.3417 |

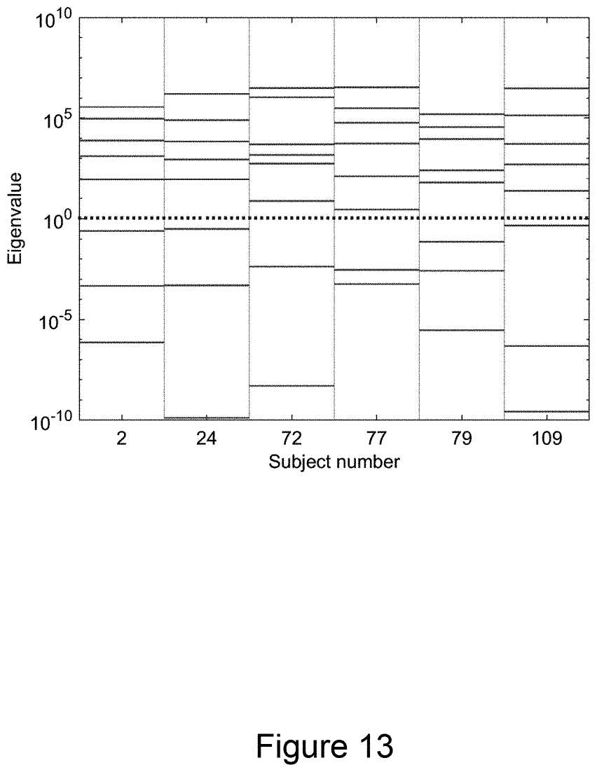

FIG. 13 shows eigenvalues of the Fisher information matrix (FIM) on a log scale for the selected subjects. The FIM is numerically calculated using dimensionless increments at the parameters corresponding to a least squares fit to the experimental spectrum. Of the 22 possible eigenvalues, roughly 7 correspond to zero, at least to the numerical accuracy of the eigenvalue estimation routine. Typically 7 of the remaining 15 are too small (relative to the largest eigenvalue) to be reliably calculated. The roughly uniform distribution of the eigenvalues on a log scale is a characteristic of a sloppy model. The blue dotted line delineates the separation of identifiable (above the dotted line) from unidentifiable (below the dotted line) regimes. Thus ˜5 parameter combinations are usually identifiable, suggesting that the 22-parameter model can be described using 5 or 6 effective parameters.

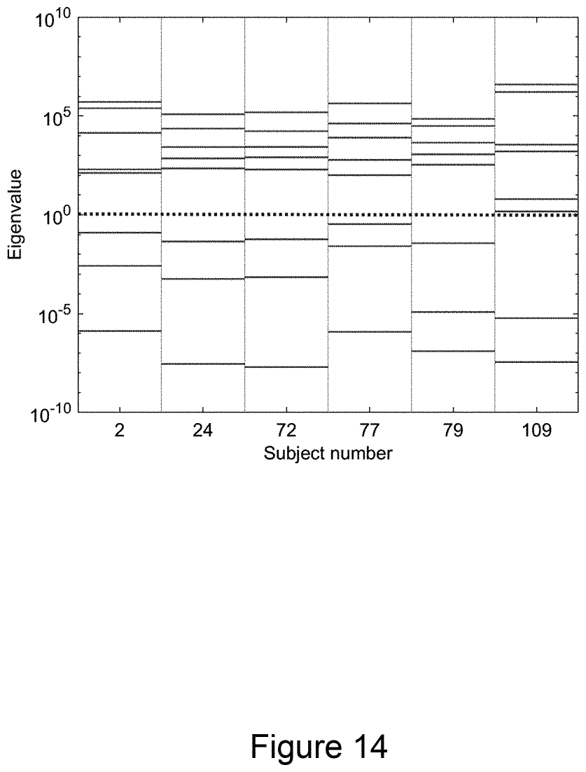

FIG. 14 shows a corresponding analysis for the MCMC method, where FIM eigenspectra are calculated around the best fit found from maximum likelihood optimization for each subject's spectrum.

In the cases shown in FIGS. 13 and 14, the eigenvalues are spread over many decades with approximately uniform spacing over a log scale. This indicates that the neural population model is sloppy. A comparison of these eigenspectra across different subjects suggests that there are usually ˜5 identifiable parameter combinations for each subject.

The larger FIM eigenvalues define eigenvector directions corresponding to identifiable parameter combinations. To understand the parameters that contribute the most to each (identifiable) eigenvector we compute the angular distance between each parameter direction and a given eigenvector. The closer the angular distance to 0 or 180 degrees, the greater the parameter contributes to the parameter combination and thus the more identifiable that parameter.

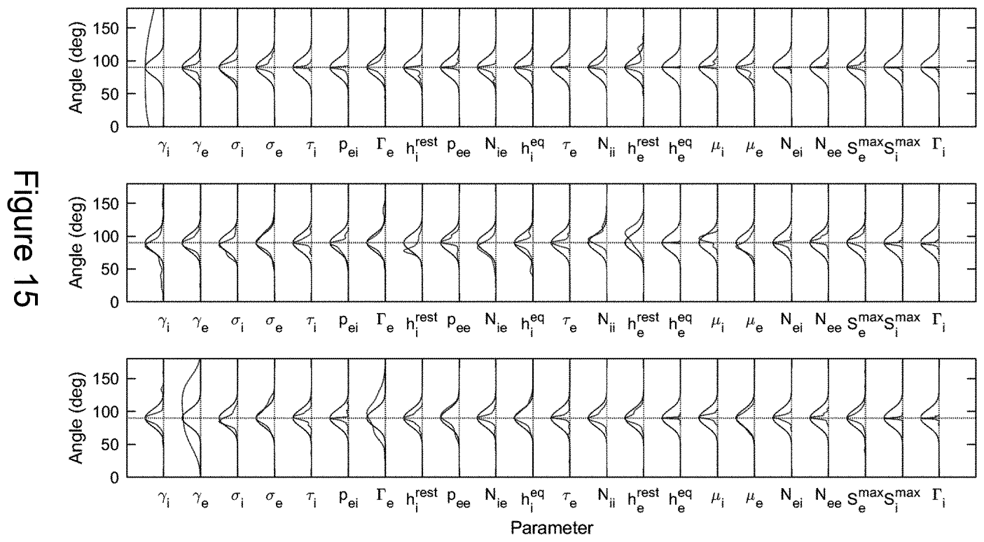

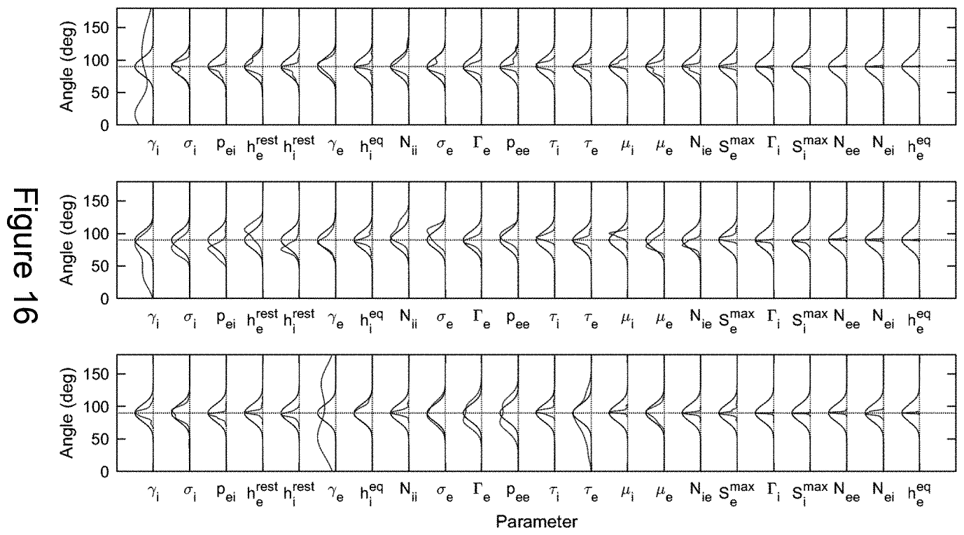

FIG. 15 shows the components of the eigenvectors corresponding to the first, second and third eigenvalues based on LS best fitting. Alignment of the leading eigenvectors relative to each parameter. Values of 0 and 180 degrees represent perfect alignment (maximum contribution) whereas 90 degrees represents orthogonality (no contribution). To compare the 82 subjects, results are presented as angular distributions (red lines). The first row is for the largest eigenvalue, the second row for the second-large eigenvalue, etc. The blue lines show the expected angular distributions for a randomly pointed vector in the 22-dimensional parameter space, illustrating how these are most likely to be orthogonal to any parameter direction. The angles are the inverse cosines of the direction cosines of the vectors. The distributions indicate that the parameters γᵢ and (to a lesser extent) γₑ may play significant roles in determining the spectral form in their own right. The remaining parameters appear largely in complicated combinations. FIG. 16 shows a corresponding distribution of the eigenvectors based on ML best fits.

The results shown in FIGS. 15 and 16 again illustrate that the γᵢ parameter is significant. It has the greatest contribution to the stiffest parameter direction, showing that it is identifiable. Interestingly, the postsynaptic potential rate constant of the excitatory population, γₑ dominates the third stiffest parameter combination. This indicates that it may also play an identifiable role in driving system dynamics, though to a lesser extent than γᵢ.

In the evaluation described above, the apparatus 100 produced a determinative parameter set consisting of 5 model parameter combinations which can be identified that characterise the alpha peak features of the input experimental EEG data. Of these parameters, the postsynaptic potential rate constant γᵢ is determined to be the most significant (i.e. identifiable). This is supported the qualitative analysis presented above, where there are only ˜5 identifiable eigenvalues in the FIM spectrum (indicating that the number of effective parameters in our model is only about 5).

Some insight into the role of the determinative parameters can be obtained by further examining the modelled spectra, such as by considering its derivatives in the directions of the leading eigenvectors of the Fisher information matrix. In broad terms, for most subjects, it appears that the leading eigenvector (effectively the rate parameter) is related to variation in the location of the alpha peak; the second eigenvector is related to the height of the alpha peak compared to the overall background level and (somewhat more loosely) the third eigenvector is associated with the width of the alpha peak. The remaining combinations do not appear to be related to easily identifiable features of the spectrum. Importantly, although each eigenvector could have contributions from all 22 of the original parameters, the leading 3 eigenvectors appear to be influenced by just a few of these. This indicates that, even though γᵢ is the only parameter that is clearly identifiable, a subset of the other parameters is being constrained as well. This suggests that the essential character of the alpha peak—its position, height and width—is determined by only a few of the original parameters, and in particular by γᵢ.

The identification of the postsynaptic inhibitory rate constant γᵢ as the determinative parameter for indicating brain function have clinical significance in the context of surgical anaesthesia. Specifically, the majority of agents used to induce a state of surgical anaesthesia are thought to function by altering the time course of postsynaptic inhibition. Being able to determine γᵢ, the postsynaptic inhibitory rate constant, by fitting model parameters to EEG data provides real-time physiological insights into the functional effect of anaesthesia. That is, by monitoring γᵢ aspects of the brain function of an individual can be inferred (e.g. a state of surgical anaesthesia) which would otherwise need to be derived from direct observation EEG signals of the individual (and subsequent analysis of the alpha-rhythm feature) which may not be possible (e.g., in the surgical setting).

In view of the above, the apparatus 100 can be configured to utilise measures of γᵢ to quantify the effect that anaesthetic agents have on a constitutive aspect of brain function. That is, by estimating the value of γᵢ from EEG data of a given subject, the subject's brain activity can be measured or monitored for clinical purposes.

In some embodiments, the apparatus 100 is configured to measure brain activity in a subject, by: receiving EEG data representing EEG measurements of electrical brain activity of the subject; and fitting parameters of a neural population model to the EEG data to determine corresponding values of said parameters, including one or more values of an inhibitory postsynaptic potential rate constant (γᵢ) that is representative of states of brain responsiveness, an increase in γᵢ relative to a reference value indicating an enhanced state of brain responsiveness, and a decrease in γᵢ relative to the reference value indicating a decrease in the state of brain responsiveness.

Continuous real-time monitoring of brain activity in a subject can be achieved by repeatedly determining a plurality of values (i.e. estimates) for γᵢ based on received EEG data obtained from the subject, and the application of a fitting process (as described above) to fit the neural population model to the EEG data.

The reference value can be determined in various ways according to particular configurations of the NAC 102. In some configurations, the reference value is a single numerical value determined according to the application of the brain activity state measurement process. The reference value may be constant, or may vary according to the type of sedation or anaesthetic treatment administered to the subject. The determination of the γᵢ value as above or below the reference value may be interpreted differently depending on the application. For example, for the purpose of monitoring the depth of sedation of an unstimulated, a decrease in γᵢ relative to the reference value can indicate an decrease in brain activity or wakefulness in the subject.

In some configurations, the reference value is a predetermined value representative of brain activity in a control subject or a plurality of control subjects. The control subject set may be determined based on the application of the brain function monitoring or measurement process (e.g., to account for particular characteristics of the surgical procedure, such as a level of sedation, and/or the subjects of the procedure). In other configurations, the reference value is determined by classification, and/or mathematical analysis. For example, the neural population model can be fitted to EEG data obtained from the subject in a state of wakefulness or awareness to determine a relevant reference value to measure their brain activity state during a subsequent surgery.

In some configurations, the reference value is obtained from a range of numerical values. For example, a reference interval may be specified by a minimum reference value and a maximum reference value, where the estimated γᵢ is compared to one or both of the minimum and maximum reference values to determine a corresponding indication of measured brain activity of the subject. In such configurations, there may be multiple reference values for the purpose of indicating whether the estimated γᵢ falls within a particular reference interval (e.g., separate intervals for Stage I-IV levels of sedation, as described below).

The apparatus 100 can be applied to measure brain activity to monitor the depth of sedation in an unstimulated subject, wherein a decrease in γᵢ relative to the reference value indicates a decrease in responsiveness or wakefulness in the subject. The reference value can be determined as described above according to the configuration of the apparatus. In some configurations, the reference value is representative of brain activity in a control subject or plurality of control subjects at a level of sedation selected from the group consisting of: minimal sedation (Stage I), conscious sedation (Stage II); deep sedation (Stage III); and medullary depression (Stage IV).

Sedation levels are typically used to qualify the responsiveness of a subject, and the state of one or more vital functions (such as airway reflexes, spontaneous ventilation, and cardiovascular function), in response to the administration of a sedative type drug. Under minimal Sedation (Stage I) subjects respond normally to verbal commands, and where airway reflexes, and ventilatory and cardiovascular functions are unaffected. Moderate Sedation (Stage II, or “Conscious Sedation”) is a depression of consciousness during which subjects respond purposefully to verbal commands, either alone or accompanied by light tactile stimulation. Deep sedation (Stage III) is a depression of consciousness during which subjects: cannot be easily aroused but respond purposefully following repeated or painful stimulation; may experience difficulty independently maintaining ventilatory function; and usually maintain cardiovascular function. Medullary depression (Stage IV) is a loss of consciousness during which subjects: are not arousable, even by painful stimulation; may experience difficulty independently maintaining ventilatory function (such that positive pressure ventilation of the airway may be required because of depressed spontaneous ventilation or drug-induced depression of neuromuscular function); and may experience impaired cardiovascular function.

The apparatus 100 can be configured to monitor depth of sedation while the resting subject is undergoing a surgical procedure, and/or where the subject has been anesthetized. In some applications, the apparatus 100 can be used to administer anaesthesia to a subject, by: administering an initial amount of anaesthetic to the subject; measuring brain activity in the subject by receiving EEG data from the subject, and determining a measured value of γᵢ by fitting the EEG data to a neural population model; determining that the subject requires additional anaesthesia once the γᵢ decreases relative to the reference value; and administering additional anaesthesia to the subject requiring additional anaesthesia.

For example, with reference to the levels of sedation described above, it may be desired to maintain a subject in a state of deep sedation during a surgical operation. An increase in γᵢ to a value above the reference value (or interval) corresponding to the state of deep sedation may indicate that the subject is waking up. In this case, additional anaesthesia is administered to maintain the desired sedation. However, if too much anaesthesia is administered then the subject may enter medullary depression as indicated by an decrease in γᵢ relative to the reference value. The brain function of the subject while anesthetized can therefore be monitored in real-time, where the measured γᵢ values are used to automatically control the administration of anaesthetic over a period of time (e.g., for the duration of a surgical procedure) according to the feedback delivery process of FIG. 3b.

Many modifications will be apparent to those skilled in the art without departing from the scope of the present invention.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A computer-implemented process for measuring brain activity of a subject, including:
(i) receiving electroencephalogram (EEG) data representing EEG measurements of electrical brain activity of the subject; and
(ii) fitting parameters of a neural population model to the EEG data to determine corresponding values of said parameters, including one or more values of an inhibitory postsynaptic potential rate constant (γᵢ);
wherein γᵢ is representative of states of brain responsiveness, an increase in γᵢ relative to a reference value indicating an enhanced state of brain responsiveness, and a decrease in γᵢ relative to the reference value indicating a decrease in the state of brain responsiveness.

2. The process of claim 1, further including the step of:
(iii) repeating steps (i) and (ii) in real-time to determine values of γᵢ for respective times to provide real-time monitoring of the brain responsiveness of the subject.

3. The process of claim 2, wherein the EEG signals are measured during sedation of an unstimulated subject, and the determined values of γᵢ for respective times quantify the depth of sedation of the subject, wherein a decrease in γᵢ relative to the reference value indicates an decrease in brain responsiveness or wakefulness of the unstimulated subject.

4. The process of claim 3, wherein the unstimulated subject is being anesthetized.

5. The process of claim 3, wherein the unstimulated subject is undergoing a surgical procedure.

6. The process of claim 1, wherein the reference value is a predetermined measurement of γᵢ in a control subject or a plurality of control subjects.

7. The process of claim 1, wherein the reference value is a value of γᵢ determined by fitting the neural population model to EEG measurements of the subject in a state of wakefulness or awareness.

8. The process of claim 3, wherein the reference value is a predetermined value of γᵢ that is representative of brain activity in a control subject or plurality of control subjects at a level of sedation selected from a group consisting of: minimal sedation (Stage I), conscious sedation (Stage II), deep sedation (Stage III) and medullary depression (Stage IV).

9. The process of claim 1, wherein an anaesthetic has been administered to the subject, and the process includes:
(i) comparing a value of γᵢ to the reference value; and
(ii) determining whether the subject requires additional anaesthesia on the basis of said comparing; and
(iii) only if it is determined that the subject requires additional anaesthesia, then: administering additional anaesthesia to the subject.

10. The process of claim 8, wherein the subject is undergoing a surgical procedure.

11. The process of claim 9, wherein the additional anaesthesia is delivered by an automated anaesthetic delivery system.

12. The process of claim 9, wherein the reference value is a predetermined value of γᵢ representative of brain activity in a control subject or a plurality of control subjects.

13. The process of claim 9, wherein the reference value is a value of γᵢ determined by fitting the neural population model to EEG measurements of the subject in a state of wakefulness or awareness.

14. The process of claim 9, wherein the reference value is a predetermined value of γᵢ that is representative of brain activity in a control subject or plurality of control subjects at a level of sedation selected from a group consisting of: minimal sedation (Stage I), conscious sedation (Stage II), deep sedation (Stage III) and medullary depression (Stage IV).

15. The process of claim 1, wherein the one or more values of γᵢ quantify alpha-rhythmic features of the brain activity of the subject.

16. A system for measuring brain activity in a subject, including:
at least one network interface configured to receive data from a communications network;
at least one computer processor; and
a memory coupled to the at least one computer processor and storing instructions that, when executed by the at least one computer processor, cause the at least one computer processor to execute the brain activity measurement process of claim 1.

17. An apparatus for measuring brain activity in a subject, including:
at least one computing device having a neural population model fitting component configured to:
(i) receive EEG data representing EEG measurements of electrical brain activity of the subject; and
(ii) fit parameters of a neural population model to the EEG data to determine one or more values of said parameters, including one or more values of an inhibitory postsynaptic potential rate constant (γᵢ);
wherein γᵢ is representative of states of brain responsiveness, an increase in γᵢ relative to a reference value indicating an enhanced state of brain responsiveness, and a decrease in γᵢ relative to the reference value indicating a decrease in the state of brain responsiveness.

18. The apparatus of claim 17, wherein the neural population model fitting component is configured to execute the brain activity measurement process of claim 2.

19. The apparatus of claim 17, including an EEG monitoring component configured to:
generate the EEG data by processing EEG signals obtained from one or more electrodes; and
transmit the EEG data to the neural population model fitting component.

20. One or more computer-readable storage media having stored thereon instructions that, when executed by at least one processor of a computing system or device, cause the at least one processor to execute the process of claim 1.
